(19) United States
(12) Patent Application Publication
(10) Pub. No.: US 2020/0323581 A1
(43) Pub. Date: Oct. 15, 2020

(54) ENERGY TREATMENT INSTRUMENT

(71) Applicant: OLYMPUS CORPORATION, Tokyo (JP)

(72) Inventor: Yuji SAKAI, Kodaira-shi (JP)

(73) Assignee: OLYMPUS CORPORATION, Tokyo (JP)

(21) Appl. No.: 16/909,449

(22) Filed: Jun. 23, 2020

Related U.S. Application Data
(63) Continuation of application No. PCT/JP2018/035356, filed on Sep. 25, 2018.

(30) Foreign Application Priority Data
Dec. 25, 2017 (JP) 2017-248395

(51) Int. Cl. A61B 18/14

(52) U.S. Cl. CPC A61B 18/1477; A61B 2018/00494; A61B 2018/1425

(57) ABSTRACT

An energy treatment instrument includes: an insertion section, a base, a needle section, an output section, and a cover portion. The needle section includes a tip end projecting in a first direction relative to the base. The output section is configured to output energy to an outside of the needle section when supplied with energy. The cover portion includes an end portion which is movable relative to the base. The cover portion is configured to move between a first position and a second position. In the first position, the cover portion is configured to protect the tip end of the needle section and the output section. In the second position, the end portion of the cover portion is evacuated from the tip end of the needle section and the output section.

### CROSS-REFERENCE TO RELATED APPLICATIONS

This application is a Continuation Application of PCT Application No. PCT/JP2018/035356, filed Sep. 25, 2018 and based upon and claiming the benefit of priority from prior Japanese Patent Application No. 2017-248395, filed Dec. 25, 2017, the entire contents of all of which are incorporated herein by reference.

### FIELD

The present invention relates to an energy treatment instrument.

### BACKGROUND

For example, U.S. Pat. No. 5,823,197 discloses a treatment instrument in which a needle-like probe is provided on an energy delivery device. The treatment instrument can output energy from the probe into a mucous membrane of the inferior turbinate, in a state in which the probe is pierced in the inferior turbinate.

Jpn. Pat. Appln. KOKAI Publication No. 2002-28166 discloses a technology in which an electrode for high-frequency treatment that is inserted into the cavitas nasi can project and retreat relative to a distal end of a sheath (insertion section). The needle electrode can be prevented from coming in contact with a biological tissue other than a treatment target part.

### BRIEF SUMMARY OF THE INVENTION

According to one aspect of the present invention, an energy treatment instrument includes: an insertion section, a base, a needle section, an output section, and a cover portion. A longitudinal axis is defined in the insertion section. The base is provided on a distal side along the longitudinal axis of the insertion section. The needle section includes a tip end projecting in a first direction relative to the base. The output section is provided toward a side of the base with respect to the tip end of the needle section. The output section is configured to output energy to an outside of the needle section when supplied with energy: The cover portion includes an end portion which is movable relative to the base. The cover portion is configured to move between a first position and a second position. In the first position, the end portion of the cover portion is disposed in a position which is equal to the tip end of the needle section or in a position which projects from the tip end in the first direction. In the first position, the cover portion is configured to protect the tip end of the needle section and the output section. In the second position, the end portion of the cover portion is evacuated from the tip end of the needle section and the output section.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view illustrating a treatment system according to each of first to fourth embodiments including modifications;

FIG. 2 is a view illustrating a cross section in a state in which a needle section is protected by a cover portion, and a schematic configuration of a handle, in an energy treatment instrument of the treatment system according to the first embodiment;

FIG. 3 is a view illustrating a cross section in a state in which the needle section is exposed from the cover portion, and a schematic configuration of the handle, in the energy treatment instrument according to the first embodiment;

FIG. 4 is a schematic view of an end effector of the energy treatment instrument, as viewed in a direction of an arrow IV in FIG. 2 and FIG. 3;

FIG. 5 is a schematic view illustrating an energy output section provided on a probe of the needle section, and illustrating a denaturing region of a biological tissue at a time when energy is output from the energy output section;

FIG. 6A is a schematic view illustrating a state in which an end effector of the energy treatment instrument according to the first embodiment and an insertion section of an endoscope are inserted into the inferior turbinate of a treatment target in the cavitas nasi cavity through the external nostril, vestibulum nasi and inferior meatus;

FIG. 6B is a schematic cross-sectional view illustrating a state in which tip ends and energy output sections of the needle section shown in FIG. 3 are disposed in a shallow layer of the lamina propria mucosae through a mucosal epithelial layer of the inferior turbinate;

FIG. 7 is a schematic view illustrating the energy output sections provided on the probes of the needle section in an end effector according to a first modification of the first embodiment, and a denaturing region of the biological tissue, which is different from the denaturing region shown in FIG. 5, at a time when energy is output from the energy output sections;

FIG. 8 is a schematic view illustrating the energy output sections provided on the probes of the needle section in an end effector according to a second modification of the first embodiment, and a denaturing region of the biological tissue, which is different from the denaturing regions shown in FIG. 5 and FIG. 7, at a time when energy is output from the energy output sections;

FIG. 9 is a view illustrating a cross section in a state in which a needle section is protected by a cover portion, and a schematic configuration of a handle, in an energy treatment instrument according to a second embodiment;

FIG. 10 is a view illustrating a cross section in a state in which the needle section is exposed from the cover portion, and a schematic configuration of the handle, in the energy treatment instrument according to the second first embodiment;

FIG. 11 is a schematic view of an end effector of the energy treatment instrument, as viewed in a direction of an arrow XI in FIG. 9 and FIG. 10;

FIG. 12 illustrates a modification of the end effector of the energy treatment instrument shown in FIG. 11;

FIG. 13 is a schematic view illustrating a state in which the needle section is protected by the cover portion, when viewed in a direction of arrow XIII in FIG. 14, in an energy treatment instrument according to a third embodiment;

FIG. 14 is a schematic view illustrating a state in which the needle section is protected by the cover portion, when viewed in a direction of arrow XIV in FIG. 13, in the energy treatment instrument according to the third embodiment;

FIG. 15 is a schematic view illustrating a state in which the needle section is exposed from the cover portion, when viewed in a direction of arrow XV in FIG. 16, in the energy treatment instrument according to the third embodiment;

FIG. 16 is a schematic view illustrating a state in which the needle section is exposed from the cover portion, when viewed in a direction of arrow XVI in FIG. 15, in the energy treatment instrument according to the third embodiment;

FIG. 17 is a schematic view illustrating a state in which the needle section is protected by the cover portion, when viewed in a direction of arrow XVII in FIG. 18, in an energy treatment instrument according to a modification of the third embodiment;

FIG. 18 is a schematic view illustrating a state in which the needle section is protected by the cover portion, when viewed in a direction of arrow XVIII in FIG. 17, in the energy treatment instrument according to the modification of the third embodiment;

FIG. 19 is a schematic view illustrating a state in which the needle section is exposed from the cover portion, when viewed in a direction of arrow XIX in FIG. 20, in the energy treatment instrument according to the modification of the third embodiment;

FIG. 20 is a schematic view illustrating a state in which the needle section is exposed from the cover portion, when viewed in a direction of arrow XX in FIG. 19, in the energy treatment instrument according to the modification of the third embodiment;

FIG. 21 is a schematic view illustrating a state in which the needle section is protected by the cover portion, when viewed in a direction of arrow XXI in FIG. 22, in an energy treatment instrument according to a fourth embodiment;

FIG. 22 is a schematic view illustrating a state in which the needle section is protected by the cover portion, when viewed in a direction of arrow XXII in FIG. 21, in the energy treatment instrument according to the fourth embodiment;

FIG. 23 is a schematic view illustrating a state in which the needle section is exposed from the cover portion, when viewed in a direction of arrow XXIII in FIG. 24, in the energy treatment instrument according to the fourth embodiment; and

FIG. 24 is a schematic view illustrating a state in which the needle section is exposed from the cover portion, when viewed in a direction of arrow XXIV in FIG. 23, in the energy treatment instrument according to the fourth embodiment.

### DETAILED DESCRIPTION

Hereinafter, embodiments for carrying out the present invention will be described with reference to the accompanying drawings.

### First Embodiment

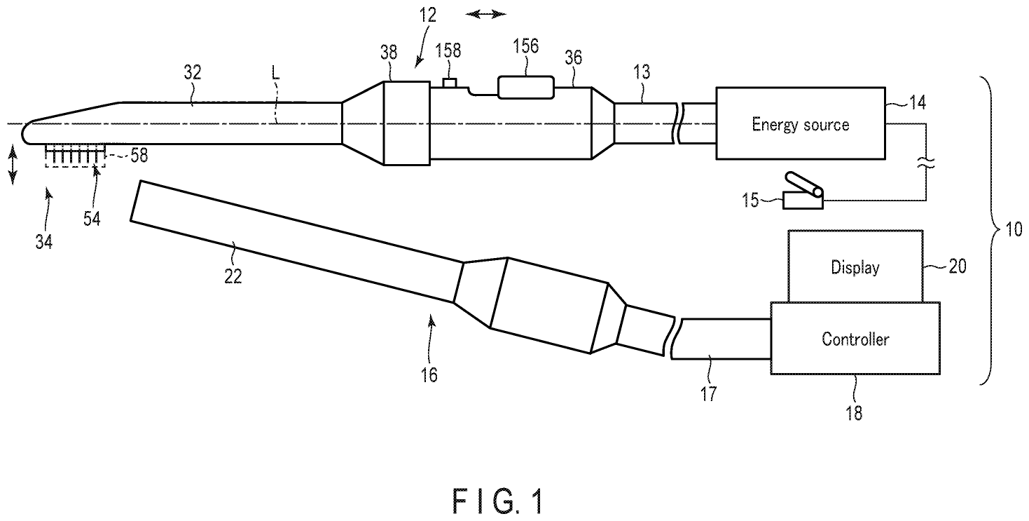

A first embodiment will be described with reference to FIG. 1 to FIG. 6B.

A treatment system 10 illustrated in FIG. 1 is used for treating the cavitas nasi, in particular, the inferior turbinate. The treatment system 10 includes an energy treatment instrument 12 and an energy source 14. The treatment system 10 of the embodiment includes an endoscope 16 and a controller 18 having a function as a power source, in addition to the energy treatment instrument 12 and energy source 14. The controller 18 includes, for example, a processor. The endoscope 16 is controlled by the processor of the controller 18. As the treatment system 10, the endoscope 16 and controller 18 are not necessarily required.

Electric power is supplied to the endoscope 16 via a cable 17 from the controller 18 which is used, for example, as a power source. The endoscope 16 captures an image of a position opposed to, for example, a distal end of an insertion section 22, and the captured image is displayed on a display 20. The insertion section 22 of the endoscope 16 may be formed to such rigidity as to as to keep the shape of the insertion section 22, or may be formed have such flexibility as to allow bending as appropriate.

The energy treatment instrument 12 includes an insertion section 32 in which a longitudinal axis L is defined, and an end effector (treatment section) 34. The energy treatment instrument 12 includes a handle 36 which is provided on a proximal side of the insertion section 32. A proximal portion of the insertion section 32 is coupled to the handle 36 which is grasped by a surgeon.

It is preferable that the energy treatment instruction 12 includes a rotary knob (rotary section) 38 which is inserted between the insertion section 32 and the handle 36 and is rotatable around the longitudinal axis L of the insertion section 32. The rotary knob 38 may be provided on the insertion section 32.

It is preferable that the rotary knob 38 is formed as one piece with the insertion section 32. When the rotary knob 38 is rotated relative to the handle 36 around the longitudinal axis L of the insertion section 32, the insertion section 32 is rotated relative to the handle 36 in the same direction as the rotational direction of the rotary knob 38 around the longitudinal axis L of the insertion section 32.

The energy source 14 is connected to the handle 36 via a cable 13. A switch 15 is connected to the energy source 14. Aside from being connected to the energy source 14, the switch 15 may be provided, for example, on the insertion section 32, handle 36 or rotary knob 38.

When the switch 15 is pushed, energy is transmitted from the energy source 14 to an energy output section (output section) 56 through a base 52 (to be described later) of the end effector 34. In the present embodiment, the energy output section 56 is described as being a high-frequency electrode through which high-frequency current is passed, but various kinds of energy may be used for treatment, such as by using a heater which generates heat. In addition, the energy output section 56 may be configured such that a heater is disposed on the high-frequency electrode and, thereby, the energy treatment instrument 12 can simultaneously perform a treatment using high-frequency current and a treatment using the heat of the heater.

The energy source 14 may be provided on the handle 36. In this case, energy is transmitted from the energy source 14, which includes a battery (not shown) provided in the handle 36, to the energy output section (output section) 56 through the insertion section 32 and the base 52 (to be described later) of the end effector 34.

The switch 15 may be configured to transmit a signal to the energy source 14 in interlock with the position of a cover portion (cover) 58 (to be described later), the position of a moving rod 82 (to be described later), and/or the position of an operation element 156 (to be described later). For example, when the operation element 156 is positioned in a first end portion 154a of a slot 154 (to be described later) and the cover portion 58 is located in a first position (to be described later), energy may be prevented from being transmitted from the energy source 14 to the end effector 34 even when the switch 15 is operated. In addition, when the operation element 156 is positioned in a second end portion 154b of the slot 154 and the cover portion 58 is located in a second position (to be described later), energy may be transmitted from the energy source 14 to the end effector 34 by the operation of the switch 15.

The end effector 34 is provided on a distal portion of the insertion section 32. In the present embodiment, the insertion section 32 and the end effector 34 are formed as one piece. Thus, the insertion section 32 and end effector 34 can rotate as one piece, relative to the handle 36, in a manner to follow the operation of the rotary knob 38 around the longitudinal axis L.

A lock, which prevents an unintended rotation of the insertion section 32 relative to the handle 36, may be provided between the handle 36 and the rotary knob 38, or between the handle 36 and the insertion section 32. The lock may be implemented by applying frictional force between the handle 36 and rotary knob 38 or between the handle 36 and insertion section 32, or may be implemented by providing a mechanism which prevents a movement between the handle 36 and rotary knob 38 or between the handle 36 and insertion section 32, such as by engaging the handle 36 and rotary knob 38 or the handle 36 and insertion section 32. For example, a mechanism may be provided which prevents a rotation between the handle 36 and rotary knob 38 or between the handle 36 and insertion section 32 in interlock with the pressing of the switch 15 and/or the movement of the operation element 156 which keeps the state in which the cover portion 58 is in a second position (to be described later).

The insertion section 32 is formed, for example, in a pipe shape. The insertion section 32 may be formed of a proper material. For example, a metallic material, which has an outer peripheral surface coated with an electrically insulating material, is used for the insertion section 32.

A so-called malleable material, which can be bent in a proper shape by a load of pressure, may be used for that portion of the insertion section. 32, which is located at a position between a proximal end of the end effector 34 and the handle 36. The insertion section 32 is bent before a treatment, for example, in accordance with the shape of the nose of each patient, and the bent shape can be kept during the treatment. Thus, in the state in which the end effector 34 on the distal side of the insertion section 32 is set in a proper direction relative to the insertion section 32, the end effector 34 can keep its direction during the treatment.

Since the end effector 34 and insertion section 32 are inserted in the patient's narrow cavity, there is a case in which proper external force acts on the end effector 34 and insertion section 32 from a wall surface in the cavitas nasi. In such a case, the insertion section 32, which is formed of the malleable material, have proper resistance to the external force from, for example, the wall surface in the body cavity. Thus, although the insertion section 32 allows proper bending, the insertion section 32 is prevented from being suddenly bent to a large degree. Accordingly, the end effector 34 on the distal side of the insertion section 32 can keep the state in which the end effector 34 is set in the proper direction relative to the insertion section 32.

Besides, it is preferable that the insertion section 32 includes a bending section having a similar structure to a bending section of an insertion section of a publicly known endoscope that can be inserted into, for example, the stomach or large intestine. In this case, the end effector 34 may be made to access the vicinity of a treatment target while properly and actively bending the bending section in accordance with the shape in the cavitas nasi. In addition, by properly bending the bending section, the end effector 34 on the distal side of the insertion section 32 is set in a proper direction relative to the insertion section 32, and this state is kept. Note that, for example, the handle 36 may be provided with a lock which prevents an unintended rotation of the bending section of the insertion section 32 during the treatment. The lock may operate in interlock with, for example, the pressing of the switch 15 and/or the movement of the operation element 156 which keeps the state in which the cover portion 58 is in a second position (to be described later). The lock may be interlocked with the above-described mechanism which prevents a rotation between the handle 36 and rotary knob 38 or between the handle 36 and insertion section 32.

In the present embodiment, the base 52, needle section 54, output section 56 and cover portion 58 of the end effector 34 are preferably pointed in a direction deviating from the longitudinal axis L of the insertion section 32 (i.e. a direction crossing the longitudinal axis L). In this embodiment, for the purpose of simple description, it is assumed that the end effector 34 is pointed in a direction perpendicular to the longitudinal axis L. The direction in which the end effector 34 is set relative to the insertion section 32 is not limited to the direction perpendicular to the longitudinal axis L, but may be set as appropriate.

A housing 50 of the end effector 34 is provided on the distal side of the insertion section 32. It is preferable that the housing 50 is formed as one piece with the distal portion of the insertion section 32. Thus, like the insertion section 32, it is preferable that the housing 50 has an outer peripheral surface coated with an electrically insulating material. It is preferable that a distal end 51 of the housing 50 is formed in an obtuse shape. Thus, when the distal end 51 of the housing 50 is passed through a passage from the entrance toward the treatment target, the passage is protected.

The end effector 34 includes a base (energy delivery device) 52 formed in a plate shape or block shape, a needle section 54, and an energy output section 56 provided in the needle section 54. In the present embodiment, the end effector 34 includes a cover portion 58 which protects the needle section 54 in the passage from the entrance toward the treatment target. The cover portion 58 is provided outside the base 52 and is movable relative to the base 52. By the movement in a predetermined direction, the cover portion 58 can advance and retreat (can move) between a first position in which the cover portion 58 protects a biological tissue from a tip end 104 (to be described later) and energy output section 56 of the needle section 54, and a second position in which the tip end 104 and energy output section 56 of the needle section 54 can treat the biological tissue. In the present embodiment, it is preferable that the cover portion 58 is movable in a direction crossing the longitudinal axis L, in particular, in a direction perpendicular to the longitudinal axis L.

It is preferable that the cover portion 58 is formed of an electrically insulating resin material. In this embodiment, a description is given of an example in which the cover portion 58 is provided outside the needle section 58.

The housing 50 includes a recess-shaped inner peripheral surface (recessed surface) 50a. The base 52 is provided on the distal side along the longitudinal axis L of the insertion section 32, and is fixed to the housing 50. Specifically, the base 52 is fixed to the inner peripheral surface 50a of the housing 50. The needle section 54 is supported on that side of the base 52, which is opposite to the part fixed to the inner peripheral surface 50a of the housing 50. The needle section 54 refers to a region where a bundle of a plurality of needle-shaped probes (micro-needles) 102 (to be described later), or a plurality of probes 102 are included.

The insertion section 32 is provided with at least a pair of electrical paths (transmission paths). The base 52 is electrically connected to the energy source 14 via lead wires (not shown) and/or a structural member or the like of the insertion section 32. For example, the insertion section 32 is electrically connected to the energy output sections 56, and may be used as a part of the transmission path for transmitting energy which is supplied from the energy source 14 to the energy output sections 56. For example, a moving rod 82 provided inside an inner peripheral surface 32a of the insertion section 32 is electrically connected to the energy output sections 56, and may be used as a part of the transmission path for transmitting energy which is supplied from the energy source 14 to the energy output sections 56. In addition, the base 52 is used as a delivery device which transmits energy to the energy output sections 56 provided in the needle section 54.

The needle section 54 includes a plurality of probes 102. The probes 102 are provided on the distal side along the longitudinal axis L of the insertion section 32. Each probe 102 projects in a first direction relative to the base 52, and can be pierced into a biological tissue. Each probe 102 includes a tip end (needle point) 104 at its distal end. Thus, the needle section 54 includes tip ends 104 projecting from the base 52, and is pierced into the biological tissue from the tip ends 104.

Each probe 102 includes a basal portion 106 which is supported on the base 52. In the present embodiment, it is preferable that, between the basal portion 106 and tip end 104 of each probe 102, a portion excluding the energy output section 56 has electrical insulation properties.

It is preferable that the respective probes 102 are straight and parallel with each other. The extending direction of each probe 102 agrees with the direction (first direction) in which the end effector 34 is directed. The extending direction (first direction) of each probe 102 is, for example, perpendicular or substantially perpendicular to the longitudinal axis L. The extending direction of each probe 102 is not limited to the direction perpendicular or substantially perpendicular to the longitudinal axis L, and may be set as appropriate. Although each probe 102 is preferably formed to be straight from the basal portion 106 to tip end 104, as described above, each probe 102 may be bent as appropriate.

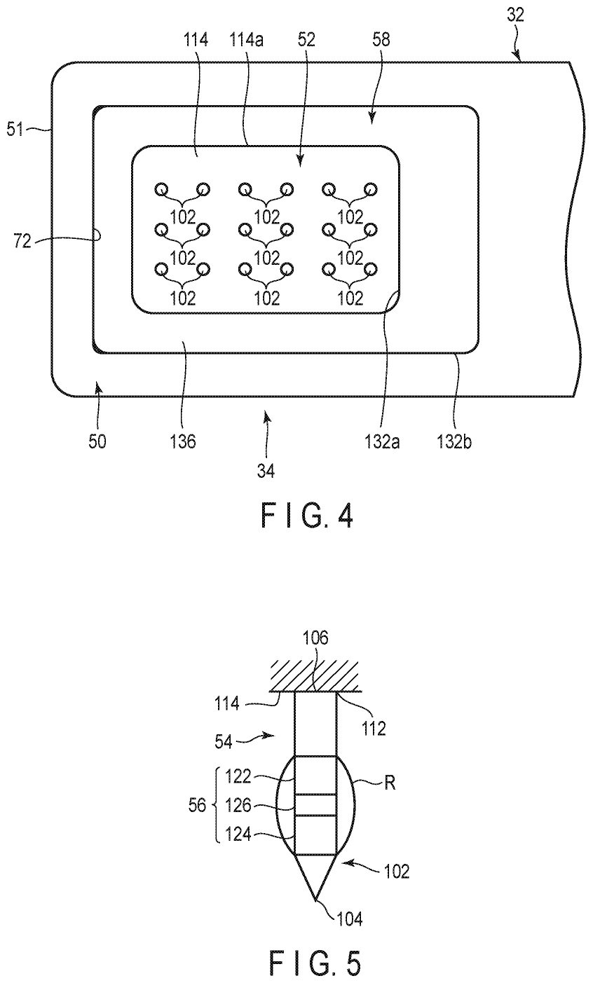

The base 52 includes a support portion 112 which supports the basal portions 106 of the probes 102 of the needle section 54. As illustrated in FIG. 4, in the support portion 112, for example, the probes 102 are arranged in a lattice shape at proper intervals. The density of probes 102 in the needle section 54 is properly set, for example, based on the size of a treatment region (a region indicated by sign R in FIG. 5) in the energy output section 56 of each probe 102. The base 52 includes a defining surface 114 which defines a length (projection length) of each probe 102 of the needle section 54 between the tip end 104 of the probe 102 and the defining surface 114 of the base 52. For the purpose of simple description, in the present embodiment, it is assumed that the defining surface 114 is a planar surface and the extending direction of each probe 102 is perpendicular to the defining surface 114.

It is preferable that the lengths (projection lengths) of the respective probes (micro-needles) 102 relative to the defining surface 114 of the base 52 are identical or substantially identical. The magnitudes of the length, diameter, etc. of each probe 102 are set based on a material and a treatment target, and also the position of a second end face (end portion) 136 of the cover portion 58. For example, it is preferable that the outside diameter of each probe 102 is about 200 μm. It is preferable that the length of each probe 102 is set such that, for example, a piercing depth from the surface of a mucosal epithelial layer MEL (to be described later) to the energy output section 56 is about 0.05 mm to 0.8 mm, although the length of each probe 102 depends on the positional relationship with the cover portion 58.

The housing 50 includes a guide 72 which guides the movement of the cover portion 58 relative to the base 52 and needle section 54. The guide 72 is provided outside the base 52. Thus, the cover portion 58 is provided outside the base 52. In this embodiment, the cover portion 58 is movable in a direction parallel to or substantially parallel to the extending direction of the probes 102 which extend straight from the base 52. The extending direction of the probes 102 is a direction crossing the longitudinal axis L of the insertion section 32. The cover portion 58 is movable in a direction crossing the longitudinal axis L of the insertion section 32. In other words, the second end face (end portion) 136 of the cover portion 58 is movable in the direction in which the needle section 54 projects. Accordingly, in the housing 50, the cover portion 58, which is provided on the outer periphery of the base 52 and needle section 54, is supported so as to be movable relative to the housing 50, base 52 and needle section 54.

The end effector 34 includes an urging body 74 which can move the cover portion 58 along the guide 72 relative to the base 52 and needle section 54. For example, the urging body 74 is provided between the cover portion 58 and the inner peripheral surface 50a of the housing 50.

In the present embodiment, for example, a plurality of coil springs 74a are used as the urging body 74. One end of each coil spring 74a is supported on the inner peripheral surface 50a of the housing 50. The other end of each coil spring 74a is supported on a first end face (end edge) 134 (to be described later) of the cover portion 58. The coil spring 74a of this embodiment urges the first end face 134 of the cover portion 58 such that the first end face 134 approaches the inner peripheral surface 50a of the housing 50. As the urging body 74, a rubber member may be sued in place of the coil springs 74a.

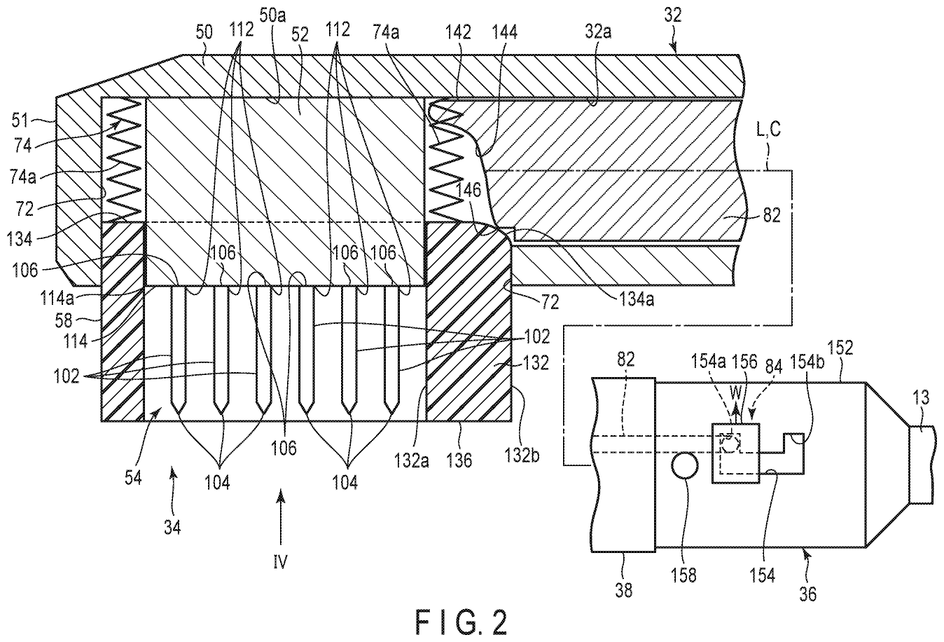

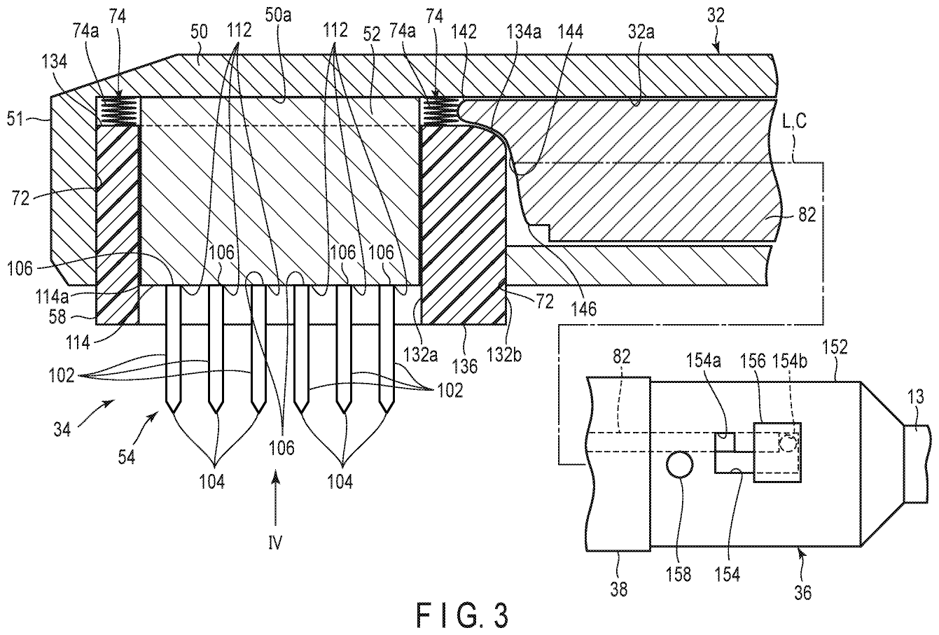

In the present embodiment, the energy treatment instrument 12 includes a lock mechanism 84 which is provided on the handle 36 and holds the position of the cover portion 58 relative to the base 52. In the lock mechanism 84, for example, the operation element 156 is held by being disposed in either of end portions 154a and 154b of a slot 154. Thus, in the energy treatment instrument 12 of this embodiment, the cover portion 58 is locked in a first position illustrated in FIG. 2, and is locked in a second position illustrated in FIG. 3. Hence, in this embodiment, the lock mechanism 84 is provided on the handle 36, and holds the position of the cover portion 58 relative to the base 52 and needle section 54.

The lock mechanism 84 may be formed on the insertion section 32 in which the moving rod 82 is included. Thus, it should suffice if the lock mechanism 84 is provided on at least one of the handle 36 and moving rod 82.

In the present embodiment, the cover portion 58 is provided outside an outer edge 114a of the defining surface 114. The cover portion 58 includes a cover body 132. In the present embodiment, the cover body 132 is described as having a cylindrical shape (an annular-shaped transverse cross section). However, the shape of the cover body 132 is not limited to the cylindrical shape, and the cover body 132 may have some other proper transverse cross-sectional shape such as a substantially C-shape or U-shape.

The cover body 132 has an inner peripheral surface 132a and an outer peripheral surface 132b. The cover body 132 has a first end face 134 which is opposed to the inner peripheral surface 50a on the depth side of the housing 50, and a second end face (a reference surface or a reference edge) 136 which is located on the opposite side to the first end face 134. The first end face 134 and second end face 136 may be planar surfaces or curved surfaces. Although the second end face 136 of the cover body 58 is described here as being a surface, the second end face 136 may not be a surface if the second end face 136 is formed as an end portion which is, for example, curved in the direction of projection. The second end face (end portion) 136 can advance and retreat (can move) in a predetermined direction (specifically, the direction of projection of the needle section 54) relative to the base 52, and is used as a reference surface (reference edge) which defines a positional relationship with the tip ends 104 of the probes 102 of the needle section 54. The second end face 136 of the cover portion 58 is formed in the direction in which the needles section 54 projects with respect to the defining surface 114 of the base 52, and is formed to be continuous with distal ends of the inner peripheral surface (wall surface) 132a and outer peripheral surface (wall surface) 132b of the cover body 132.

As illustrated in FIG. 2, the second end face 136 of the cover portion 58 is disposed in a position (on the projection direction side) which is equal to the tip end 104 of the needle section 54 or projects from the tip end 104. This position is defined as “first position”. Thus, when the cover portion 58 is in the first position, the second end face 136 of the cover portion 58 is disposed in a position which is equal to the tip end 104 of the probe 102 of the needle section 54, or in a position which is farther from the defining surface 114 of the base 52 than the tip end 104. In this manner, when the cover portion 58 is in the first position, the second end face (end portion) 136 of the cover portion 58 is disposed in a position which is equal to the tip end 104 of the needle section 54, or in a position which projects from the tip end 104, thereby protecting the tip ends 104 and output sections 56 of the needle section 54. Specifically, when the cover portion 58 is in the first position, the cover portion 58 surrounds and covers the outside of the needle section. 54 in such a state that the tip ends (needle points) 104 of the probes 102 are not exposed.

As illustrated in FIG. 3, the second end face 136 of the cover portion 58 is disposed in a position (on the base 52 side) which is closer to the base 52 than the tip ends 104 and energy output sections 56 of the needle section 54. This position is defined as “second position”. Thus, the second end face 136 of the cover portion 58 is disposed in a position which is closer to the defining surface 114 of the base 52 than the tip ends 104 and energy output sections 56 of the probes 102 of the needle section 54. In this manner, when the cover portion 58 is in the second position, the second end face (end portion) 136 of the cover portion 58 is evacuated from the tip ends 104 and output sections 56 of the needle section 54. In other words, when the cover portion 58 is in the second position, the tip ends (needle points) 104 and energy output sections 56 of the probes 102 project from the second end face (end portion) 136 with respect to the cover portion 58. At this time, the tip ends (needle points) 104 and energy output sections 56 of the probes 102 are exposed from the cover portion 58. Thus, when the cover portion 58 is in the second position, the end effector 34 is capable of piercing the tip ends 104 and energy output sections 56 of the needle section 54 into the biological tissue.

In this manner, the cover portion 58 can advance and retreat (can move) between the first position (see FIG. 2) and the second position (see FIG. 3). Thus, when the cover portion 58 moves from the first position to the second position, the tip ends (needle points) 104 and energy output sections 56 of the probes 102 can project from the second end face (end portion) 136 of the cover portion 58.

In the present embodiment, the second position where the tip ends 104 of the probes 102 are exposed, the second end face 136 of the cover portion 58 is located closer to the tip ends 104 of the needle section 54 than the defining surface 114 of the base 52. Although not illustrated, in the second position where the tip ends 104 of the probes 102 are exposed, the second end face 136 of the cover portion 58 may be located farther from the tip ends 104 of the needle section 54 than the defining surface 114 of the base 52.

As illustrated in FIG. 5, the energy output section 56 is provided in a predetermined position between the tip end 104 and basal portion 106 of each probe 102. In the present embodiment, the position of the energy output section 56 is adjusted such that the energy output section 56 reaches a lamina propria mucosae through a mucosal epithelial layer. The energy output section 56 can output energy to the outside of the needle section 54 by being supplied with energy from the energy source 14 via the cable 13. In the present embodiment, the energy output section 56 includes a first electrode 122 with electrical conductivity, a second electrode 124 with electrical conductivity, and a spacer 126 with electrical insulation properties, which is provided between the first electrode 122 and second electrode 124. The first electrode 122 and second electrode 124 are electrically connected to the energy source 14 via independent electrical paths (lead wires), respectively. Note that, in each probe 102 of the needle section 54, a part at a position outside the energy output section 56 has electrical insulation properties. In addition, the energy output section 56 can pass high-frequency current between the first electrode 122 and second electrode 124 via a biological tissue. Thus, each energy output section 56 can locally denature, for example, a tissue of a region R shown in FIG. 5 in the biological tissue.

The energy source 14 can monitor the state of a mucosal tissue by acquiring information of impedance or the like of the mucosal tissue near each energy output section 56, by using each energy output section 56 as a sensor. Thus, by using the present system 10, the coagulation state of the mucosal tissue can be understood as in the publicly known art. In addition, by monitoring the information of impedance or the like, the energy source 14 can recognize whether the energy output section 56 is in contact with a biological tissue. It is thus preferable that the energy source 14 outputs energy of such a degree as not to affect the biological tissue which is in contact with the energy output section 56, immediately before outputting such energy as to affect the biological tissue which is in contact with the energy output section 56. Therefore, the energy source 14 can output such energy as to affect the biological tissue which is in contact with the energy output section 56, after judging whether the energy output section 56 of each probe 102 is exactly in contact with the biological tissue.

The energy source 14 may be capable of adjusting electric current which is passed through each energy output section 56, based on biological information of each of the individual energy output sections 56.

In the present embodiment, as illustrated in FIG. 3, when the cover portion 58 is in the second position, the second end face 136 of the cover portion 58 is positioned closer to the tip ends 104 of the probes 102 than the defining surface 114 of the base 52. Thus, when the cover portion 58 is in the second position, the end effector 34 is capable of piercing the tip ends 104 and energy output sections 56 of the needle section 54 into the biological tissue. In addition, the position of the energy output section 56 relative to the second end face 136 of the cover portion 58 at a time when the cover portion 58 is in the second position corresponds to a distance for reaching the lamina propria mucosae through the mucosal epithelial layer.

As described above, depending on the state of design, there may be a case in which when the cover portion 58 is in the second position, the defining surface 114 of the base 52 is closer to the tip ends 104 of the probes 102 than the second end face 136 of the cover portion 58. In this case, the projection length of the energy output section 56 relative to the defining surface 114 of the base 52 is a distance for reaching the lamina propria mucosae through the mucosal epithelial layer.

In the present embodiment, the first end face 134 of the cover portion 58 includes an inclined surface 134a. The inclined surface 134a is formed at a position on the proximal side along the longitudinal axis L in the first end face 134 of the cover portion 58. The inclined surface 134a is formed as a planar surface or a curved surface.

In this embodiment, the energy treatment instrument 12 includes the moving rod (moving body) 82 which is provided between the handle 36 and end effector 34 and is movable along the longitudinal axis L. The moving rod 82 is formed of, for example, the same malleable material as the insertion section 32, and is bent in a manner to follow the movement of the insertion section 32.

A distal portion of the moving rod 82 includes a projection portion 142 which abuts on, or approaches, the base 52; an inclined surface 144 which is continuous with the projection portion 142; and an abutment surface 146 which is continuous with the inclined surface 144 and is abutted on the inclined surface 134a of the cover portion 58. It is preferable that the moving rod 82 and projection portion 142 are electrically insulated, for example, by applying coatings with electrical insulation properties to outer surfaces thereof.

The inclined surface 144 is formed as a planar surface or a curved surface. The inclined surface 144 is inclined to a plane perpendicular to the longitudinal axis L. The projection portion 142 is located at a position close to the inner peripheral surface 50a in the housing 50, relative to the inclined surface 144.

A proximal portion of the moving rod 82 extends to the handle 36. The handle 36 includes a main body 152 which is formed, for example, in a cylindrical shape; the slot (groove) 154 formed in the main body 152; and the operation element 156 coupled to the proximal portion of the moving rod 82 through the slot 154. The slot 154 is formed along the longitudinal axis L. The slot 154 is provided with, as parts of the lock mechanism 84, a first end portion 154a and a second end portion 154b which are continuous. The first end portion 154a is formed in a position on the distal side along the longitudinal axis L in the slot 154. The second end portion 154b is formed in a position on the proximal side along the longitudinal axis L in the slot 154. The slot 154 including the first end portion 154a and second end portion 154b is formed, for example, as a substantially U-shape as a whole. The operation element 156 is disposed in the first end portion 154a of the slot 154, when the cover portion 58 is kept, i.e. locked, in the first position illustrated in FIG. 2. The operation element 156 is disposed in the second end portion 154b of the slot 154, when the cover portion 58 is kept, i.e. locked, in the second position illustrated in FIG. 3.

As illustrated in FIG. 2, when the cover portion 58 is in the first position, it is preferable that the operation element 156 of the handle 36 is located at one end 154a of the slot 154 and is urged in a width direction W which is perpendicular to the longitudinal axis L. The abutment surface 146 of the distal portion of the moving rod 82 is abutted on the inclined surface 134a of the first end face 134 of the cover portion 58. Thus, the first end face 134 of the cover portion 58 is located apart from the inner peripheral surface 50a of the housing 50.

As illustrated in FIG. 3, when the cover portion 58 is in the second position, it is preferable that the operation element 156 of the handle 36 is located at the other end 154b of the slot 154 and is urged in the width direction W which is perpendicular to the longitudinal axis L. The inclined surface 144 of the distal portion of the moving rod 82 is abutted on the inclined surface 134a of the first end face 134 of the cover portion 58. Thus, the first end face 134 of the cover portion 58 is located near the inner peripheral surface 50a of the housing 50.

An indicator 158 is provided on the handle 36. In the present embodiment, for example, the indicator 158 emits light, only while energy from the energy source 14 is being output. In particular, it is preferable that the indicator 158 emits light, only while energy is being supplied to each energy output section 56 and a biological tissue is being treated by each energy output section 56.

Next, the function of the treatment system 10 according to the present embodiment will be described. In particular, a description will be given of an example in which a mucous membrane of the nose is treated by using the energy treatment instrument 12. Note that, in other cases, too, a treatment may be performed by using the energy treatment instrument 12.

For instance, allergic rhinitis is a disease of many patients worldwide. This disease includes seasonal allergic rhinitis generally called “hay fever”, and year-round allergic rhinitis with house dust or a pet being an allergen. Main symptoms of the allergic rhinitis include nasal congestion, snivel, sneezing, and itching. The first choice of a treatment for each symptom is, basically, a medication, but an operative therapy may be applicable to a patient with a serious illness.

There are various operative therapies, which include, for example, (1) an operative therapy aiming at reducing and denaturing a mucous membrane of the nose, (2) corrective surgery of nasal cavity which aims at improving the degree of nasal airflow, and (3) an operative therapy aiming at shutting off neurotransmission.

Here, for example, such a case is mainly described that the (1) operative therapy aiming at reducing and denaturing a mucous membrane of the nose is performed by using the energy treatment instrument 12. At present, it has begun to be recognized that the mucous membrane of the inferior turbinate is a main place of a nasal allergic reaction. As regards the allergic rhinitis, it has begun to be understood that a treatment of properly denaturing the mucous membrane of the inferior turbinate, such as by cauterization or coagulation, is effective.

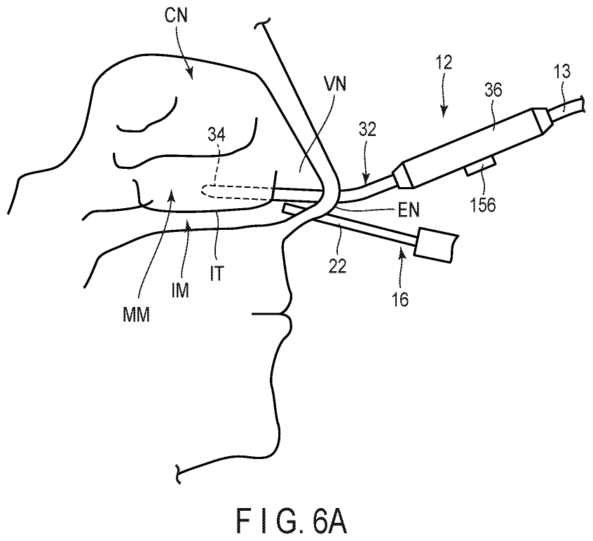

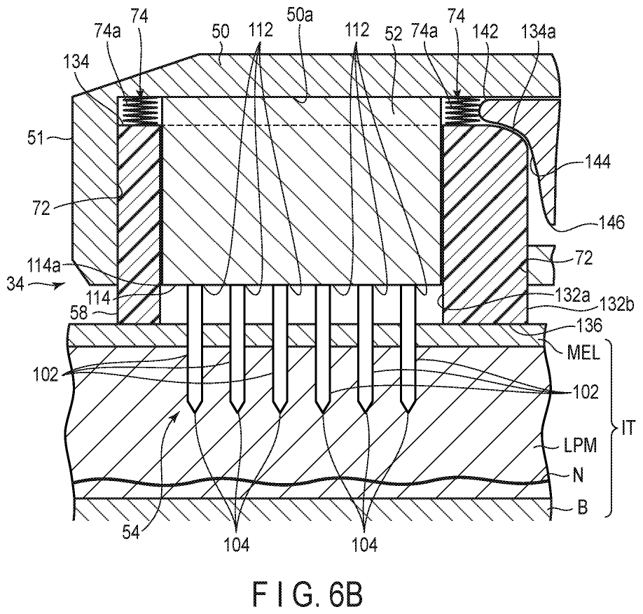

A surgeon sets the cover portion 58 of the end effector 34 of the energy treatment instrument 12 in the first position (see FIG. 2) in advance. As illustrated in FIG. 6A, the surgeon inserts the end effector 34 of the energy treatment instrument 12 into the inferior turbinate IT of a treatment target in the cavitas nasi CN through a patient's external nostril EN, vestibulum nasi VN and, for example, inferior meatus IM. The surgeon disposes, when necessary, a distal end of the insertion section 22 of the endoscope 16 at a position where a part of the inner wall of the cavitas nasi CN and a part of the end effector 34 of the energy treatment instrument 12 can be observed. When the endoscope 16 is used, a part of the inner wall of the cavitas nasi CN and a part of the end effector 34 of the energy treatment instrument 12 are displayed on the display 20 by the endoscope 16.

Then, the surgeon puts the second end face 136 of the cover portion 58 of the end effector 34 into contact with the mucous membrane of the inferior turbinate IT. Note that the posterior nasal nerve N1 (not shown) on the rear side of the inferior turbinate IT may be accessed via not the inferior meatus IM but the middle meatus MM.

As illustrated in FIG. 2, in the end effector 34 of the energy treatment instrument 12 according to the present embodiment, the tip end 104 of each probe 102 is positioned closer to the defining surface 114 of the base 52 than the second end face 136 of the cover portion 58. Thus, when the end effector 34 of the energy treatment instrument 12 is inserted toward the inferior turbinate IT of the treatment target from the cavitas nasi CN, the tip end 104 of each probe 102 is prevented from abutting on the wall surface in the range from the cavitas nasi CN of the patient to the inferior turbinate IT of the treatment target. In addition, since the tip end 104 of each probe 102 is prevented from abutting on the wall surface in the range from the cavitas nasi CN of the patient to the inferior turbinate IT of the treatment target, a load is prevented from acting on each probe 102 before the treatment target is treated.

As illustrated in FIG. 6E, in the inferior turbinate IT, the mucosal epithelial layer MEL and lamina propria mucosae LPM are present from the outside toward the inside. When the surgeon puts the second end face 136 of the cover portion 58 into contact with the mucous membrane of the inferior turbinate IT, it is preferable that the tip end 104 of each probe 102 of the needle section 54 is not in contact with the surface of the mucosal epithelial layer MEL, but the tip end 104 of each probe 102 may be in contact with the surface of the mucosal epithelial layer MEL.

The surgeon moves the operation element 156 of the handle 36 from the position shown in FIG. 2 to the position shown in FIG. 3, against the urging force applied to the operation element 156. At this time, in interlock with the movement of the operation element 156, the moving rod 82 is moved toward the proximal side along the longitudinal axis L of the insertion section 32. By the urging force of the springs 74a of the urging body 74, the first end face 134 of the cover portion 58 is moved to approach the inner peripheral surface 50a of the housing 50 along the guide 72, while keeping the state in which the inclined surface 134a abuts on the inclined surface 144 of the distal portion of the moving rod 82.

When the operation element 156 is disposed in the other end 154b of the slot 154, the positions of the cover portion 58 and the moving rod 82 relative to the housing 50 are defined. Accordingly, the cover portion 58 of the end effector 34 is held in the second position. At this time, the projection length between the tip end 104 of the probe 102 and the second end face 136 of the cover portion 58 is defined. Thus, the projection length up to the energy output section 56 relative to the second end face 136 of the cover portion 58 is defined.

The surgeon keeps the state in which the second end face 136 of the cover portion 58 of the end effector 34 is put in contact with the mucous membrane of the inferior turbinate IT. Thus, as illustrated in FIG. 6B, in the state in which the second end face 136 of the cover portion 58 is abutted on the surface of the mucosal epithelial layer MEL, the tip ends 104 of the probes 102 reach the lamina propria mucosae LPM via the mucosal epithelial layer MEL. It is preferable that the tip ends 104 of the probes 102 do not reach the bone B. In addition, in particular, the energy output sections 56 are disposed in an upper layer of the lamina propria mucosae LPM, which includes a shallow layer of the lamina propria mucosae LPM. At this time, the outer peripheral surfaces of the first electrode 122, second electrode 124 and spacer 126 of the energy output section 56 illustrated in FIG. 5 are in contact with the upper layer including the shallow layer of the lamina propria mucosae LPM.

In this state, if the surgeon pushes the switch 15, the indicator 158 provided on the handle 36 is turned on, and high-frequency current is supplied from the energy source 14 to the shallow layer of the lamina propria mucosae LPM through the first electrode 122 and second electrode 124 of the energy output section 56 provided in each probe 102. The shallow layer of the lamina propria mucosae LPM, which is a tissue that is in contact with the energy output section 56, and a peripheral tissue thereof (the region indicated by sign R in FIG. 5) are denatured, for example, by being locally cauterized by high-frequency current. Note that the treatment region R of each probe 102 in the needle section 54 is changed based on the state of the tissue, the magnitude of current, etc.

Here, the energy output section 56 is used as a sensor which acquires biological information such as impedance. In addition, the energy source 14, which is electrically connected to the energy output section 56, fully recognizes the state (biological information) of the shallow layer of the lamina propria mucosae LPM, which is in contact with the energy output section 56, and the peripheral tissue thereof. Thus, while high-frequency current is being passed, the state of denaturing of the tissue, which is in contact with the energy output section. 56, and the peripheral tissue thereof is estimated.

In addition, when the energy source 14 judges that the biological information has reached a predetermined threshold, the energy source 14 automatically stops the output of energy to the tissue, which is in contact with the energy output section 56, and the peripheral tissue thereof. At this time, even in the state in which the switch 15 is pressed, the energy source 14 turns off the indicator 158. The surgeon can recognize, by the turn-off of the indicator 158, the end of the treatment, i.e. the end of the output of energy to the tissue, which is in contact with the energy output section 56, and the peripheral tissue thereof. Note that when the indicator 158 is turned off, the energy source 14 may completely stop the supply of energy or may pass such a weak level of current as not to affect the biological tissue. Specifically, when the indicator 158 is turned off, the output from the energy source 14 is automatically reduced.

As described above, the energy output section 56, which has reached the lamina propria mucosae LPM, cauterizes the shallow layer of the lamina propria mucosae LPM by passing high-frequency current. On the other hand, the region, in which a cauterizing treatment is performed by the energy output section 56, is limited to a narrow range, and such a cauterizing treatment as to cause damage on the surface of the mucosal epithelial layer MEL is prevented from being performed.

By performing such treatment, the shallow layer of the lamina propria mucosae LPM is denatured (coagulated) without causing damage on the surface of the mucosal epithelial layer MEL, and a cicatrix is formed in the shallow layer of the lamina propria mucosae LPM. Thus, a mucous membrane, which does not easily swell, is formed in the shallow layer of the lamina propria mucosae LPM of the inferior turbinate IT. Further, the occurrence of an allergic reaction is suppressed in the shallow layer of the lamina propria mucosae LPM of the inferior turbinate IT after the treatment.

By using the energy treatment instrument 12 according to the present embodiment, it is possible to prevent damage from occurring on the surface of the mucosal epithelial layer MEL. Therefore, it is possible to prevent a ciliary movement function of the surface of the mucosal epithelial layer MEL from being affected.

Since the inferior turbinate IT has a proper size, it is preferable to perform a treatment twice or more in a divided manner by the energy treatment instrument 12. The tip ends 104 of the probes 102 are pulled out from the lamina propria mucosae LPM and mucosal epithelial layer MEL of the inferior turbinate IT, and the second end face 136 of the cover portion 58 is abutted on the mucosal epithelial layer MEL in a neighboring region in the inferior turbinate IT. At this time, the tip ends 104 of the probes 102 are made to reach the lamina propria mucosae LPM via the mucosal epithelial layer MEL. Then, in the same manner as described above, the surgeon denatures a tissue, which is in contact with the energy output sections 56, and a peripheral tissue thereof, into a proper state by the supply of energy from the energy source 14.

When the surgeon largely moves the end effector 34, as in the case of pulling out the end effector 34 from the cavitas nasi CN, the surgeon moves the operation element 156 from the other end 154b toward the one end. 154a of the slot 154. At this time, the moving rod 82 advances along the longitudinal axis L of the insertion section 32. The inclined surface 144 of the moving rod 82 pushes the inclined surface 134a of the first end face 134 of the cover portion 58, against the urging force of the springs 74a of the urging body 74. In addition, the abutment surface 146 is abutted on the inclined surface 134a. Thus, the second end face 136 of the cover portion 58 is positioned farther from the defining surface 114 of the base 52 than the tip ends 104 of the probes 102. In other words, the tip ends 104 of the probes 102 are positioned closer to the defining surface 114 of the base 52 than the second end face 136 of the cover portion 58. Further, the operation element 156 is kept in the state in which the operation element 156 is disposed in the one end 154a of the slot 154. Thus, the cover portion 58 is restored from the second position shown in FIG. 3 to the first position shown in FIG. 2.

The inferior turbinate IT generally has a curved shape. Thus, there may be a case in which the surgeon wishes to adjust the direction of the end effector 34. In this case, since the surgeon needs to operate, for example, the operation element 156, there may be a case in which it is preferable that the grasping state of the handle 36 is maintained. Thus, the surgeon rotates the rotary knob 38 around the longitudinal axis L relative to the handle 36. In accordance with the rotation of the rotary knob 38, the insertion section (cylindrical body) 32, which is coupled to the rotary knob 38, and the moving rod 82 in the inside of the insertion section 32 are rotated together. Accordingly, the direction of the end effector 34 on the distal side of the insertion section 32 is adjusted in a proper state.

In addition, the operation element 156 of the handle 36 is moved to a proper position, and the shallow layer of the lamina propria mucosae LPM of the inferior turbinate IT is cauterized as needed, in the same manner as described above.

In the meantime, when the above-described (3) operative therapy aiming at shutting off neurotransmission is to be performed, for example, it may be necessary to treat a nerve N near the bone B. In many cases, the nerve N of the treatment target exists near the surface of the bone B. Thus, compared to the case of performing the above-described (1) operative therapy aiming at reducing and denaturing a mucous membrane of the nose, a depth from the surface of the mucosal epithelial layer MEL may become large. In this case, there is a possibility that the height of the cover portion 58 and/or the length of the probe 102 becomes different from the example shown in FIG. 6B, as regards the above-described (1) operative therapy aiming at reducing and denaturing a mucous membrane of the nose and the above-described (3) operative therapy aiming at shutting off neurotransmission. Thus, the treatment instrument 12 for the case of performing the treatment of the above-described (3) is different from the treatment instrument 12 for the case of performing the treatment of the above-described (1).

However, when the cover portion 58 is in the second position, the position of the energy output section 56 relative to the second end face (reference surface) 136 of the cover portion 58 or the defining surface 114 of the base 52 is determined. Thus, the depth from the surface of the mucosal epithelial layer MEL to the energy output section 56 can be made constant. Accordingly, by using high-frequency current in the energy output section 56, the nerve N in the lamina propria mucosae LPM near the surface of the bone B can exactly be cauterized. Therefore, by using the energy treatment instrument 12 according to the present embodiment, an allergic reaction in the inferior turbinate IT can be suppressed and the occurrence of rhinorrhea can be suppressed.

According to the energy treatment instrument 12 of the present embodiment, the following can be said.

By using the energy treatment instrument 12 according to the present embodiment, when the treatment target is accessed by the end effector 34, even if the probes 102 of the needle section 54 are present in the end effector 34, it is possible to protect the tip ends 104 of the probes 102 by the cover portion 58 and to protect the path to the treatment target. Thus, according to the present embodiment, there can be provided the energy treatment instrument 12 which can protect the inside of the cavity at a time of accessing the treatment target. Furthermore, at a time of accessing the treatment target by the end effector 34, the probes 102 of the needle section 54 can be protected by the cover portion 58.

Besides, in the energy treatment instrument 12 according to the present embodiment, the needle section 54 is fixed to the base 52 and does not move. Thus, a mechanical load, which is received when the needle section 54 is moved into and out of the biological tissue, may be applied to the electrical path of the energy treatment instrument 12. However, there is no need to consider a load on the electrical path due to the movement of the needle section relative to the insertion section. Therefore, according to the present embodiment, there can be provided the energy treatment instrument 12 which can reduce the load acting on the electrical path.

By using the energy treatment instrument 12, the end effector 34 of the treatment instrument 12 can be made to access, in particular, the inside of the cavitas nasi CN that is complex and narrow, such as the inferior turbinate IT having a wide range and a projecting shape, without causing damage to other tissues.

In addition, the end effector 34 of the treatment instrument 12 can be made to approach, with no invasion or low invasion, the shallow layer of the lamina propria mucosae LPM that is considered to be effective in modern treatment, without causing damage on the surface of the mucous membrane (mucosal epithelial layer MEL). Further, by using the energy treatment instrument 12, energy can be output to only the shallow layer of the lamina propria mucosae LPM, and the tissue of the shallow layer of the lamina propria mucosae LPM can be denatured. Specifically, the tissue of the shallow layer of the lamina propria mucosae LPM can be cauterized by using high-frequency current. Besides, when the cover portion 58 is in the second position, the position of the energy output section 56 relative to the second end face (reference surface) 136 of the cover portion 58 or the defining surface 114 of the base 52 is determined. Thus, the depth from the surface of the mucosal epithelial layer MEL to the energy output section 56 can be made constant. Accordingly, by using high-frequency current in the energy output section 56, the tissue of the shallow layer of the lamina propria mucosae LPM can exactly be cauterized. Therefore, by using the energy treatment instrument 12 according to the present embodiment, an allergic reaction in the inferior turbinate IT can be suppressed and the occurrence of a blocked nose, i.e. nasal congestion, can be suppressed.

In the meantime, there is a case in which the above-described (1) operative therapy aiming at reducing and denaturing a mucous membrane of the nose is performed by laser technology. This operative therapy (laser cauterization) is known as an operation method with relatively low invasion. However, in a treatment by a laser beam, since the laser beam is made to reach the lamina propria mucosae LPM through the mucosal epithelial layer MEL, damage may be caused on the mucosal epithelial layer MEL. In other words, the treatment by the laser beam may cause damage on the ciliary movement of the surface of the mucosal epithelial layer MEL. In addition, it is said that, for example, due to the damage on the surface of the mucosal epithelial layer MEL, the symptom is aggravated by the influence of the damage on the surface of the mucosal epithelial layer MEL during half a day to about one week after the operation.

In the energy treatment instrument 12 according to the present embodiment, although the probes (micro-needles) 102 are pierced into the mucosal epithelial layer MEL, energy is hardly applied to the mucosal epithelial layer MEL itself, the surface of the mucosal epithelial layer MEL is hardly damaged, and only the shallow layer of the lamina propria mucosae LPM can be denatured (coagulated) by the application of energy. In this manner, with the use of the energy treatment instrument 12 according to the present embodiment, since no damage is caused on the surface of the mucosal epithelial layer MEL, the effect of treatment can easily be obtained immediately after the surgical operation, compared to the conventional treatment means.

(First Modification)

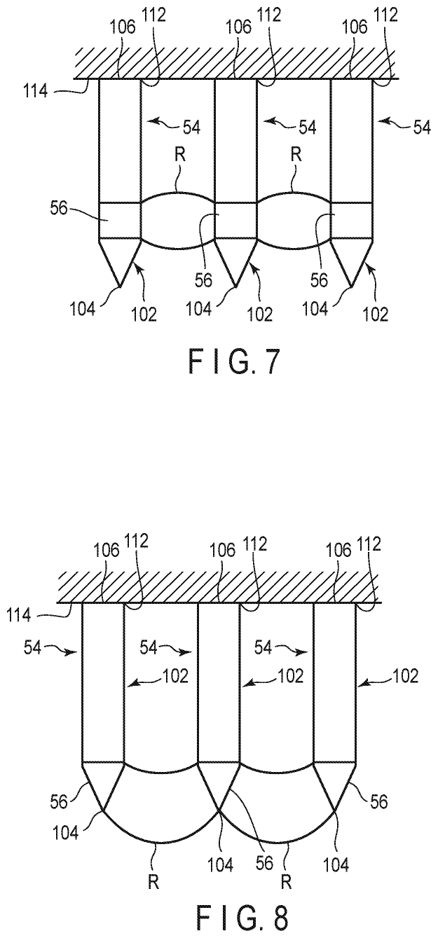

Here, referring to FIG. 7, a modification of the energy output section 56 will be described. In the example illustrated in FIG. 5, each energy output section. 56 includes the first electrode 122 and second electrode 124. In the example illustrated in FIG. 7, when an electric current is passed from the energy source 14, energy output sections 56 provided on neighboring probes 102 are used as electrodes of different polarities. Thus, for example, a region R illustrated in FIG. 7 is denatured (coagulated) by the output of energy between the energy output sections 56 of the neighboring probes 102. Accordingly, a part of the shallow layer of the lamina propria mucosae LPM is properly cauterized.

(Second Modification)

An example illustrated in FIG. 8 is a further modification of the example of the energy output section 56 shown in FIG. 7. The energy output section 56 of the example illustrated in FIG. 5 and FIG. 7 is present at the tip end 104 of the probe 102 in a position closer to the basal portion 106 than the tip end 104. The energy output section 56 of the example illustrated in FIG. 8 is present in a position including the tip end 104 of the probe 102. Even when the energy output section 56 is formed in this manner, for example, a region R illustrated in FIG. 8 is denatured by the output of energy between the energy output sections 56 of the neighboring probes 102. Accordingly, a part of the shallow layer of the lamina propria mucosae LPM is properly cauterized. In this manner, the position and range of the region R can be adjusted by the disposition or the like of the energy output section 56 provided on each probe 102.

(Third Modification)

In the first embodiment, first modification and second modification, the example in which the lamina propria mucosae LPM is treated by using high-frequency current of a so-called “bipolar system” was described. Here, an example of using a monopolar system is described.

The treatment system 10 according to this modification includes a return electrode (not shown) which is connected to the energy source 14 and is attached to a patient. The return electrode is attached, for example, to the thigh of the patient.

The energy output sections 56 of the probes 102 of the end effector 34 are electrically connected and set at an identical potential. If energy is output from the energy source 14 in this state, high-frequency current is passed through the shallow layer of the lamina propria mucosae LPM, which is in contact with the energy output section 56 provided in each probe 102, and the peripheral tissue thereof, and the current is recovered by the return electrode. In addition, of the shallow layer of the lamina propria mucosae LPM, which is in contact with the energy output section 56, and the peripheral tissue thereof, the region through which a high-density, high-frequency current is passed is cauterized.

In this manner, if the region R at a proper depth (e.g. the shallow layer of the lamina propria mucosae LPM and the peripheral tissue thereof) can be treated by passing high-frequency current to the proper depth of the biological tissue (e.g. the shallow layer of the lamina propria mucosae LPM and the peripheral tissue thereof), one of the bipolar method and the monopolar method may properly be selected as the method of passing high-frequency current in the probes 102 of the needle section 54.

### Second Embodiment

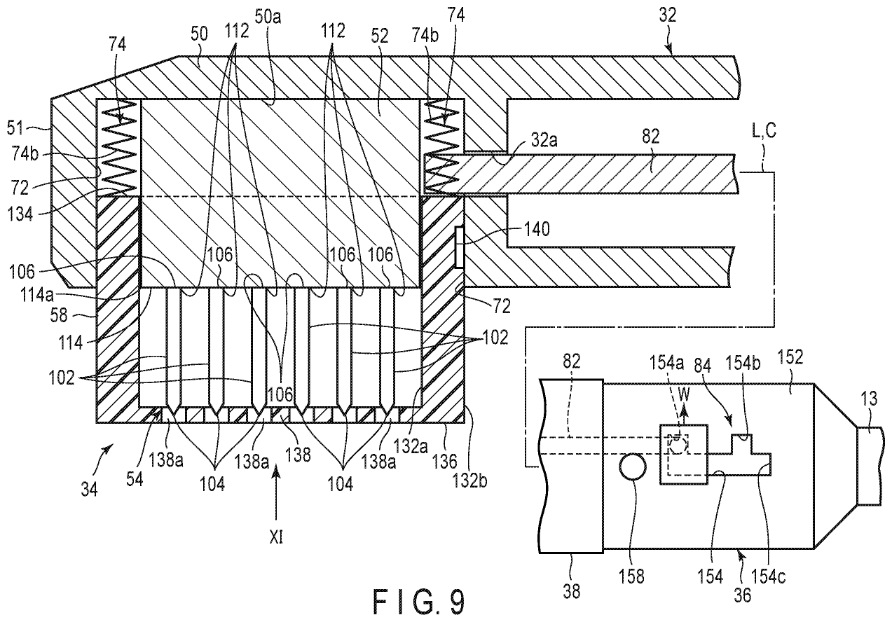

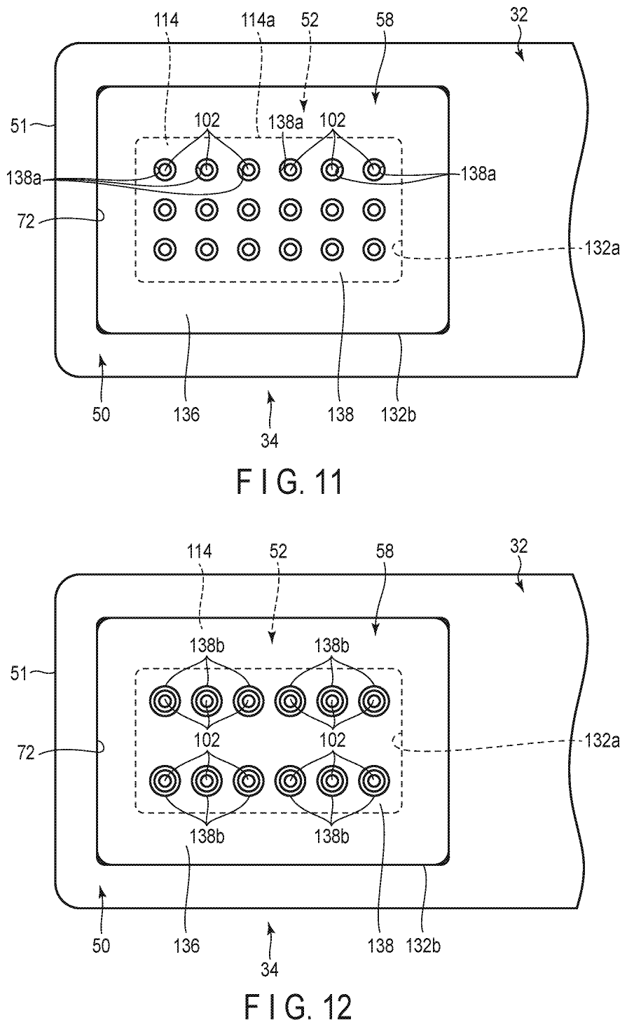

Next, a second embodiment will be described with reference to FIG. 9 to FIG. 11. This embodiment is a modification of the first embodiment including each modification. The same members or the members having the same functions as the members described in the first embodiment are denoted by like reference signs, and a detailed description thereof is omitted.

As illustrated in FIG. 9 to FIG. 11, a plate-shaped member 138 is formed on the second end face 136 of the cover portion 58. Thus, the plate-shaped member 138 moves in a manner to follow the movement of the cover portion 58 along the guide 72. Openings 138a, through which the probes 102 of the needle section. 54 are passed, are formed in the plate-shaped member 138.

In the present embodiment, for example, a plurality of coil springs 74b are used as the urging body 74. Similarly as described in the first embodiment, one end of each coil spring 74b is supported on the inner peripheral surface 50a of the housing 50. The other end of each coil spring 74b is supported on the first end face of the cover portion 58. In the present embodiment, unlike the first embodiment described above, the coil spring 74b urges the first end face 134 of the cover portion 58 in such a manner that the first end face 134 moves away from the inner peripheral surface 50a of the housing 50. As the urging body 74, a rubber member may be used in place of the coil springs 74b.

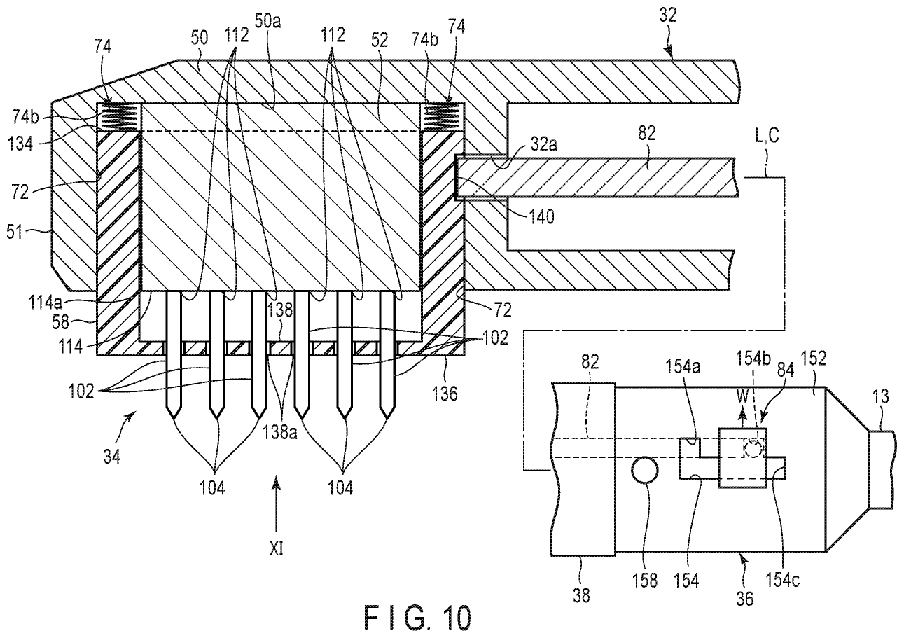

As illustrated in FIG. 9, the second end face 136 and the plate-shaped member 138 of the cover portion 58 are disposed in a position which is equal to the tip end 104 of the needle section 54, or in a position which is more on the projection direction side of the needle section 54 than the tip end 104. This position is defined as “first position”. Thus, when the cover portion 58 is in the first position, the second end face 136 of the cover portion 58 and the plate-shaped member 138 are disposed in a position which is equal to the tip end 104 of the probe 102 of the needle section 54, or in a position which is farther from the defining surface 114 of the base 52 than the tip end 104. In other words, when the cover portion 58 is in the first position, the cover portion 58 sets the tip end 104 of each probe 102 of the needle section 54 at a position closer to the base 52 than the second end face 136 and plate-shaped member 138. The cover portion 58 can be pushed in a direction toward the inner peripheral surface 50a of the housing 50. Specifically, as illustrated in FIG. 10, the cover portion 58 can set the tip end 104 and energy output section 56 of each probe 102 of the needle section 54 at a position farther from the base 52 than the second end face 136 and plate-shaped member 138 through the openings 138a of the plate-shaped member 138. At this time, the second end face 136 of the cover portion 58 and the plate-shaped member 138 are disposed in a position which is closer to the base 52 than the tip ends 104 and energy output sections 56 of the needle section 54. This position is defined as “second position”. Thus, when the cover portion 58 is in the second position, the tip ends (needle points) 104 and energy output sections 56 of the probes 102 project from the second end face (end portion) 136 and plate-shaped member 138 with respect to the cover portion 58. Therefore, when the cover portion 58 is in the second position, the end effector 34 is capable of piercing the tip ends 104 and energy output sections 56 of the probes 102 of the needle section 54 into the biological tissue.

The cover portion 58 can advance and retreat (can move) between the first position and the second position. Thus, when the cover portion 58 is in the first position, the cover portion 58 protects the passage from the entrance to the treatment target toward the treatment target from the tip end 104 of each probe 102, and protects each probe 102 for use in a treatment.

A center axis C of the moving rod 82 agrees or substantially agrees with the longitudinal axis L of the insertion section 32.

When the cover portion 58 is in the first position, a distal portion of the moving rod 82 is in contact with the first end face 134 of the cover portion 58. A recessed groove 140 is formed in an outer peripheral surface 132b of the cover portion 58. When the cover portion 58 is in the second position, the distal end of the moving rod 82 is engaged in the recessed groove 140.

In the present embodiment, the slot 154 does not have a simple U-shape, but includes an extension portion 154c which extends to the proximal side. When the cover portion. 58 is moved from the first position to the second position, and when the cover portion 58 is moved from the second position to the first position, the operation element 156 is disposed in the extension portion 154c.

Next, the function of the treatment system 10 according to the present embodiment will be described. A description of the parts described in the first embodiment is omitted unless otherwise necessary.

As illustrated in FIG. 9, when the cover portion 58 of the end effector 34 of the treatment instrument 12 is in the first position, the distal portion of the moving rod 82 is used as a stopper. Thus, even when the second end face 136 of the cover portion 58 is pushed toward the inner peripheral surface 50a of the housing 50, the movement of the cover portion 58 relative to the housing 50 is restricted. Similarly as described in the first embodiment, in the state in which the cover portion 58 is in the first position, the end effector 34 is made to access the inferior meatus IM. Then, the surgeon puts the second end face 136 of the cover portion 58 and the plate-shaped member 138 of the end effector 34 into contact with the surface of the mucosal epithelial layer MEL of the inferior turbinate IT.

As illustrated in FIG. 9, in the end effector 34 of the energy treatment instrument 12 according to the present embodiment, the tip end 104 of each probe 102 is positioned closer to the defining surface 114 of the base 52 than the second end face 136 of the cover portion 58 and the plate-shaped member 138. Thus, when the end effector 34 of the energy treatment instrument 12 is inserted toward the inferior turbinate IT of the treatment target from the cavitas nasi CM, the tip end 104 of each probe 102 is prevented from abutting on the wall surface in the range from the cavitas nasi CN of the patient to the inferior turbinate IT of the treatment target. In addition, since the tip end 104 of each probe 102 is prevented from abutting on the wall surface in the range from the cavitas nasi CN of the patient to the inferior turbinate IT of the treatment target, a load is prevented from acting on each probe 102 before the treatment target is treated.

The surgeon moves the operation element 156 of the handle 36 to the extension portion 154c on the proximal side of the slot 154 against the urging force of the elastic body from the urged state in the position shown in FIG. 9. At this time, if the surgeon pushes the second end face 136 of the cover portion 58 and the plate-like member 138 onto the mucous membrane of the inferior turbinate IT, the first end face 134 of the cover portion 58 is caused to approach the inner peripheral surface 50a of the housing 50 along the guide 72, against the urging force of the coil springs 74b of the urging body 74. Thus, the tip ends 104 of the probes 102 project, relative to the openings 138a of the plate-shaped member 138.

In this state, if the operation element 156 is disposed in the other end 154b of the slot 154, the distal end of the moving rod 82 is engaged in the recessed groove 140, as illustrated in FIG. 10. Accordingly, the cover portion 58 of the end effector 34 is held in the second position. At this time, the projection length up to the tip end 104 of the probe 102 relative to the second end face 136 of the cover portion 58 and the plate-shaped member 138 is defined. Thus, the projection length up to the energy output section 56 relative to the second end face 136 of the cover portion 58 and the plate-shaped member 138 is defined.

Since the surgeon pushes the second end face 136 of the cover portion 58 and the plate-like member 138 of the end effector 34 onto the mucous membrane of the inferior turbinate IT, the tip ends 104 of the probes 102 reach the lamina propria mucosae LPM via the mucosal epithelial layer MEL in the state in which the second end face 136 of the cover portion 58 and the plate-shaped member 138 are abutted on the mucosal epithelial layer MEL. In particular, the energy output sections 56 are disposed in the upper layer including the shallow layer of the lamina propria mucosae LPM.

If the surgeon pushes the switch 15, an electric current is caused to flow in the shallow layer of the lamina propria mucosae LPM from the energy source 14 via the energy output sections 56, and the shallow layer of the lamina propria mucosae LPM of the inferior turbinate IT is properly cauterized, as described in the first embodiment, first modification, second modification and third modification.

When the surgeon largely moves the end effector 34, as in the case of pulling out the end effector 34 from the cavitas nasi CN, if the surgeon disposes the operation element 156 from the other end 154b into the extension portion 154c of the slot 154, the distal end of the moving rod 82 is disengaged from the recessed groove 140 of the cover portion 58. Thus, the cover portion 58 is restored from the state shown in FIG. 10 to the state shown in FIG. 9 along the guide 72 by the urging force of the coil springs 74b of the urging body 74. Then, the surgeon disposes the operation element 156 from the extension portion 154c of the slot 154 into the one end 154a. At this time, the distal portion of the moving rod 82 restricts the movement of the first end face 134 of the cover portion 58 toward the inner peripheral surface 50a of the housing 50. By the coil springs 74b of the urging body 74, the cover portion 58 is prevented from dropping from the guide 72 of the housing 50. Thus, the cover portion 58 is kept in the state of the first position relative to the housing 50. In the state in which the inside of the cavitas nasi CN is protected from the needle section 54, the surgeon can move the end effector 34 of the treatment instrument 12, for example, can pull out the end effector 34 from the cavitas nasi CN.

In the present embodiment, the example was described in which the moving rod 82 can be moved by using the operation element 156 of the handle 36. However, in this embodiment, the moving rod 82 is not necessarily required. For example, by properly selecting the springs 74b, such as by selecting the strength of springs 74b and/or the number of springs 74b, the surgeon can move the cover portion 58 along the guide 72 against the urging force of the springs 74b, in the state in which the second end face 136 of the cover portion 58 of the end effector 34 is abutted on the surface of the mucosal epithelial layer MEL of the inferior turbinate IT. At this time, the energy output sections 56 provided in the probes 102 can be disposed in the shallow layer of the lamina propria mucosae LPM. At this time, by using energy, the shallow layer of the lamina propria mucosae LPM and the peripheral tissue thereof can be treated as appropriate. In addition, by moving the cover portion 58 along the guide 72 in accordance with the urging force of the springs 74b, the surgeon can dispose the tip ends 104 of the probes 102 of the needle section 54 at a position closer to the base 52 than the second end face 136 of the cover portion 58.

The cover portion 58 including the plate-shaped member 138 described in the second embodiment may be used in place of the cover portion 58 described in the first embodiment. Similarly, the cover portion 58 described in the first embodiment may be used in place of the cover portion 58 described in the second embodiment.

Although not illustrated, as described in the first embodiment, the insertion section 32 may be formed to be properly bendable.

(Modification)

In the above-described example illustrated in FIG. 9, the openings 138a are formed in the plate-shaped member 138. Alternatively, in an example illustrated in FIG. 12, cylindrical bodies 138b are provided in place of the openings (holes) 138a. The plate-shaped member 138 including the cylindrical bodies 138b can be used in place of the plate-shaped member 138 including the openings 138a described in the second embodiment, by adjusting the position of the plate-shaped member 138 relative to the cover portion 58, and adjusting the position of the end face (reference surface) in the cylindrical body 138b, which is far from the depth-side inner peripheral surface 50a of the housing 50. In addition, in the state in which the cover portion 58 is disposed in the first position, in particular, the probes 102 can individually be protected by the cylindrical bodies 138b.

### Third Embodiment

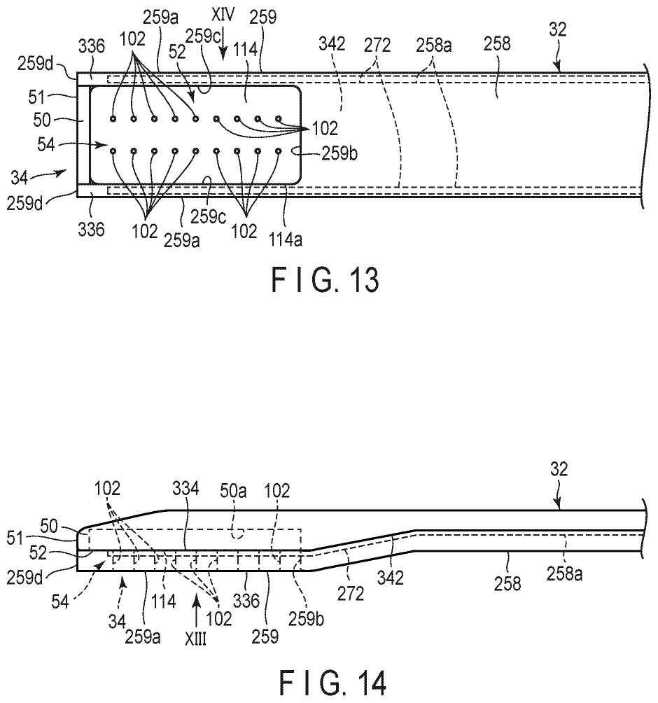

Next, a third embodiment will be described with reference to FIG. 13 to FIG. 16. This embodiment is a modification of the first embodiment including each modification and the second embodiment including each modification. The same members or the members having the same functions as the members described in the first embodiment and second embodiment are denoted by like reference signs, and a detailed description thereof is omitted.

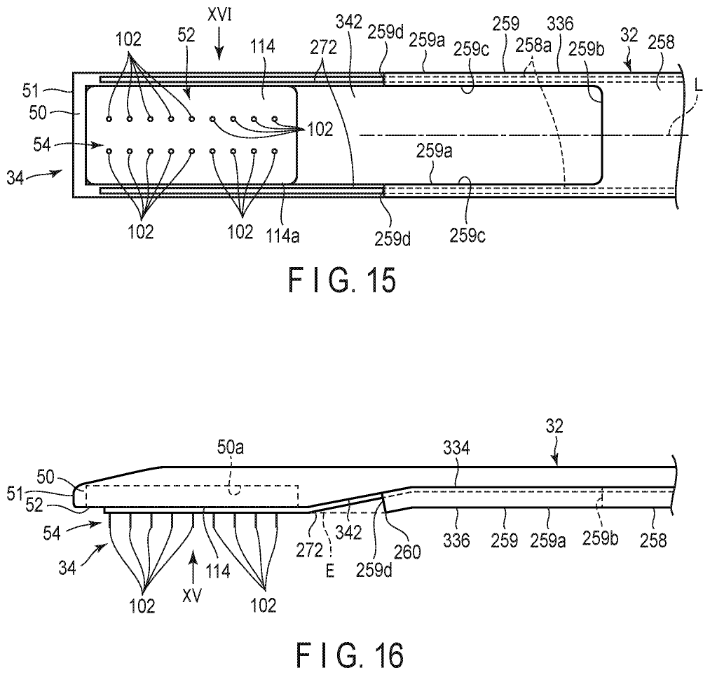

An energy treatment instrument 12 according to the present embodiment includes, like the first embodiment and second embodiment, an insertion section 32 in which a longitudinal axis L is defined, and an end effector 34. In the present embodiment, the treatment instrument 12 includes a cover portion 258. It is preferable that the cover portion 258 is formed of an electrically insulating resin material. The end effector 34 includes a base 52, a needle section 54, and energy output sections 56. The cover portion 258 is movable relative to the base 52. In the present embodiment, the cover portion 258 extends along the longitudinal axis L of the insertion section 32. The cover portion 258 is movable along the longitudinal axis 1 of the insertion section 32. Specifically, the cover portion 258 is movable between a position illustrated in FIG. 13 and FIG. 14 and a position illustrated in FIG. 15 and FIG. 16. Thus, the cover portion 258 of the present embodiment differs from the cover portion 58 described in the first embodiment and second embodiment, with respect to the movable direction and the shape.

The insertion section 32 is provided with at least a pair of electrical paths (transmission paths). The base 52 is electrically connected to the energy source 14 via lead wires (not shown) and/or a structural member or the like of the insertion section 32. For example, the insertion section 32 is electrically connected to the energy output sections 56, and may be used as a part of the transmission path for transmitting energy which is supplied from the energy source 14 to the energy output sections 56.

Guides (rails) 272 are formed on the insertion section 32 of the energy treatment instrument. 12 and the housing 50 of the end effector 34 illustrated in FIG. 13 to FIG. 16. The cover portion 258 includes guides 258a which are movable along the guides 272. A distal portion 259 of the cover portion 258 is formed in a substantially U-shape. FIG. 13 and FIG. 15 illustrate an example in which the number of guides 272 of the insertion section 32 and the housing 50 of the end effector 34 is two, but the number of guides 272 may be one, or three or more. Thus, the number of guides 258a of the cover portion 258 is adjusted based on the number of guides 272 of the insertion section 32 and the housing 50 of the end effector 34.

A proximal portion (not shown) of the cover portion 258 along the longitudinal axis L of the insertion section 32 is coupled to the operation element 156 of the handle 36 shown in FIG. 1.

In addition, when the operation element 156 is advanced to the frontmost part in the slot 154, the guides 258a of the cover portion 258 move to the distal side along the longitudinal axis L along the guides 272 of the insertion section 32 and the housing 50 of the end effector 34. Thus, the distal portion 259 of the cover portion 258 is disposed on the end effector 34. A second end face (end portion) 336 (to be described later) of the cover portion 258 is disposed in a position which is equal to the tip end 104 of the needle section 54, or in a position (projection direction side) which projects from the tip end 104. This position is defined as “first position” of the cover portion 258. Thus, when the cover portion 258 is in the first position, the second end face 336 of the cover portion 258 is disposed in a position which is equal to the tip end 104 of the probe 102 of the needle section 54, or in a position which is farther from the defining surface 114 of the base 52 than the tip end 104. In this manner, when the cover portion 258 is in the first position, the second end face (end portion) 336 of the cover portion 258 is disposed in a position which is equal to the tip end 104 of the needle section 54, or in a position which projects from the tip end 104, thereby protecting the tip ends 104 and output sections 56 of the needle section 54. Specifically, when the cover portion 258 is in the first position, the cover portion 258 surrounds and covers the outside of the needle section 54 in such a state that the tip ends (needle points) 104 of the probes 102 are not exposed.

When the operation element 156 is retreated to the rearmost position in the slot 154, the guides 258a of the cover portion 258 move to the proximal side along the longitudinal axis L along the guides 272 of the insertion section 32 and the housing 50 of the end effector 34. The second end face 336 of the cover portion 258 is evacuated from the tip ends 104 and output sections 54 of the needle section 54. At this time, the second end face 336 of the cover portion 258 is disposed in a position (base 52 side) which is closer to the base 52 than the tip ends 104 and energy output sections 56 of the needle section 54. This position is defined as “second position” of the cover portion 258. Thus, the distal portion 259 of the cover portion 258 is evacuated from the end effector 34 and is disposed in the distal portion of the insertion section 32. Therefore, the second end face 336 of the cover portion 258 is disposed in a position which is closer to the defining surface 114 of the base 52 than the tip ends 104 and energy output sections 56 of the probes 102 of the needle section 54. Specifically, when the cover portion 258 is in the second position, the tip ends (needle points) 104 and energy output sections 56 of the probes 102 project from the second end face (end portion) 336 with respect to the cover portion 258. At this time, the tip ends (needle points) 104 and energy output sections 56 of the probes 102 are exposed from the cover portion 258. Thus, when the cover portion 258 is in the second position, the end effector 34 is capable of piercing the tip ends 104 and energy output sections 56 of the needle section 54 into the biological tissue.

In addition, the cover portion 258 can advance and retreat (can move) between the first position and the second position.

In the present embodiment, the distal portion 259 of the cover portion 258 includes a pair of extension portions 259a which extend straight or substantially straight, and a proximal edge 259b which is formed in proximal portions of the extension portions 259a, is perpendicular to the longitudinal axis L and faces the distal side along the longitudinal axis L. The paired extension portions 259a include a pair of opposed surfaces 259c which are opposed to each other. When the cover portion 258 is in the first position, the needle section 54 is present between the paired opposed surfaces 259c. In addition, the needle section 54 is opposed to the proximal edge 259b.

The distal portion 259 of the cover portion 258 includes a first end face 334 which is opposed to or put in contact with the inner peripheral surface 50a of the housing 50 in which the base 52 is provided; and a second end face (reference surface (reference edge)) 336 which is located opposite to the first end face 334. Like the second end face 136 described in the first embodiment, the second end face 336 is formed as an end portion. The first end face 334 and second end face 336 may be planar surfaces or curved surfaces. As illustrated in FIG. 14, the thickness of the distal portion 259 of the cover portion 258 (the distance between the first end face 334 and second end face 336) is set such that the tip ends 104 of the probes 102 are not visually recognized when the end effector 34 is observed from a lateral side. Thus, when the cover portion 258 is in the first position, the tip ends 104 of the needle section 54 are disposed in a position which is closer to the defining surface 114 of the base 52 than the second end face (reference surface) 336.

An inclined surface 342 is formed between the proximal portion of the housing 50 of the end effector 34 and the distal portion of the insertion section 32. When the cover portion 258 is disposed in the second position, an edge portion 260 at the distal end of the cover portion 258 is, for example, located on an extension line E of the guide 272 in the end effector 34. Thus, in the case of treating the shallow layer of the lamina propria mucosae LPM by using the end effector 34, when the guides 272 are abutted on the surface of the mucosal epithelial layer MEL, the edge portion 260 at the distal end of the cover portion 258 is prevented from interfering with the surface of the mucosal epithelial layer MEL. Therefore, when a part of the lamina propria mucosae LPM and a part of the nerve N are treated by using the end effector 34, the cover portion 258, which is disposed apart from the end effector 34, scarcely becomes an obstacle.

When the position of the distal end 51 of the housing 50 and the position of distal ends 259d of the cover portion 258 agree or substantially agree along the longitudinal axis L, the position of the outer edge 114a of the defining surface 114 of the base 52 and the position of the proximal edge 259b of the cover portion 258 agree or substantially agree along the longitudinal axis L.

Next, the function of the treatment system 10 according to the present embodiment will be described. A description of the parts described in the first embodiment and/or second embodiment is omitted unless otherwise necessary.

When the treatment target is accessed, the distal portion 259 of the cover portion 258 is disposed in the position illustrated in FIG. 13 and FIG. 14, relative to the housing 50 of the end effector 34 of the energy treatment instrument 12. At this time, the second end face 336 of the cover portion 258 is disposed in a position which is farther from the defining surface 114 of the base 52 than the tip ends 104 of the probes 102 of the needle section 54. Thus, when the end effector 34 of the energy treatment instrument 12 is inserted from the cavitas nasi CN toward the inferior turbinate IT of the treatment target, the tip end 104 of each probe 102 is prevented from abutting on the wall surface in the range from the cavitas nasi CN of the patient to the inferior turbinate IT of the treatment target. In addition, since the tip end 104 of each probe 102 is prevented from abutting on the wall surface in the range from the cavitas nasi CN of the patient to the inferior turbinate IT of the treatment target, a load is prevented from acting on each probe 102 before the treatment target is treated.

By moving the operation element 156 of the handle 36 shown in FIG. 1, the surgeon moves the cover portion 258 from the first position illustrated in FIG. 13 and FIG. 14 to the second position illustrated in FIG. 15 and FIG. 16, relative to the insertion section 32. At this time, as illustrated in FIG. 15 and FIG. 16, the distal portion 259 of the cover portion 258 is evacuated from the end effector 34. Thus, in the present embodiment, the projection length up to the tip end 104 of the probe 102 relative to the guide 272 is defined. In a modification, the projection length up to the tip end 104 of the probe 102 relative to the defining surface 114 of the base 52 may be defined. In this manner, in the end effector 34 of the present embodiment, the projection length up to the energy output section 56 relative to the guide 272 or the defining surface 114 of the base 52 is defined.

The surgeon puts the guides 272 of the housing 50 of the end effector 34 and/or the defining surface 114 of the base 52 into contact with the surface of the mucosal epithelial layer MEL of the inferior turbinate IT. Thus, in the state in which the guides 272 and/or the defining surface 114 of the base 52 is in contact with the mucosal epithelial layer MEL, the tip ends 104 of the probes 102 reach the lamina propria mucosae LPM via the mucosal epithelial layer MEL. In particular, the energy output sections 56 are disposed in the upper layer including the shallow layer of the lamina propria mucosae LPM.

In addition, similarly as described in the first embodiment including each modification and the second embodiment including each modification, if the surgeon pushes the switch 15, an electric current is caused to flow in the shallow layer of the lamina propria mucosae LPM from the energy source 14 via the energy output sections 56, and the shallow layer of the lamina propria mucosae LPM of the inferior turbinate IT is properly cauterized.

The inclined surface 342 is formed near the boundary between the insertion section 32 and the end effector 34. The inclined surface 342 has a thickness (height) shown in FIG. 14 and FIG. 16, which gradually increases from the proximal side toward the end effector 34 on the distal side along the longitudinal axis L of the insertion section 32. When the cover portion 258 is in the second position, the edge portion 260 at the distal end of the cover portion 258 is located on the extension line E of the guide 272 in the end effector 34. Thus, when the cover portion 258 is in the second position, the cover portion 258 is prevented from interfering with the surface of the mucosal epithelial layer MEL.

In the distal portion 259 of the cover portion 258, a plate-shaped member, which is similar to the plate-shaped member described in the second embodiment, may be formed on the second end face (reference surface) 336. In this case, when the cover portion 258 is in the first position, the tip ends 104 of the probes 102 can be more exactly prevented from being exposed, and the probes 102 can more exactly be protected. Thus, according to the present embodiment, there can be provided the energy treatment instrument 12 which can protect the inside of the cavity when the treatment target is accessed. In addition, when the treatment target is accessed by the end effector 34, the probes 102 of the needle section 54 can be protected by the cover portion 258.

Besides, in the energy treatment instrument 12 according to the present embodiment, similarly as described in the first embodiment and the second embodiment, there is no need to consider a load on the electrical path due to the movement of the needle section relative to the insertion section. Therefore, according to the present embodiment, there can be provided the energy treatment instrument 12 which can reduce the load acting on the electrical path.

Although not illustrated, as described in the first embodiment, the insertion section 32 may be formed to be properly bendable.

(Modification)

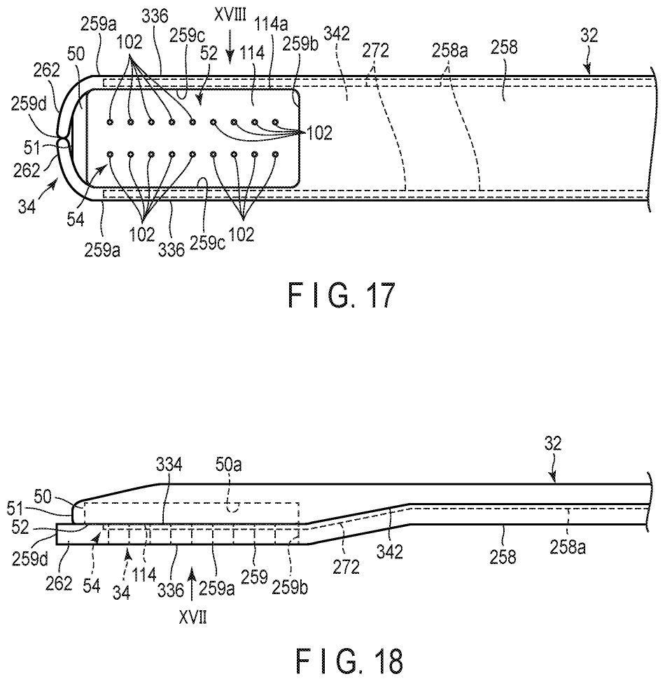

A modification of the cover portion 258 of the third embodiment will be described with reference to FIG. 17 to FIG. 20.

In the present modification, in the distal portion 259 of the cover portion 258, shape-memory portions 262 that are reformed are formed on the distal side of the paired extension portions 259a which extend straight or substantially straight.

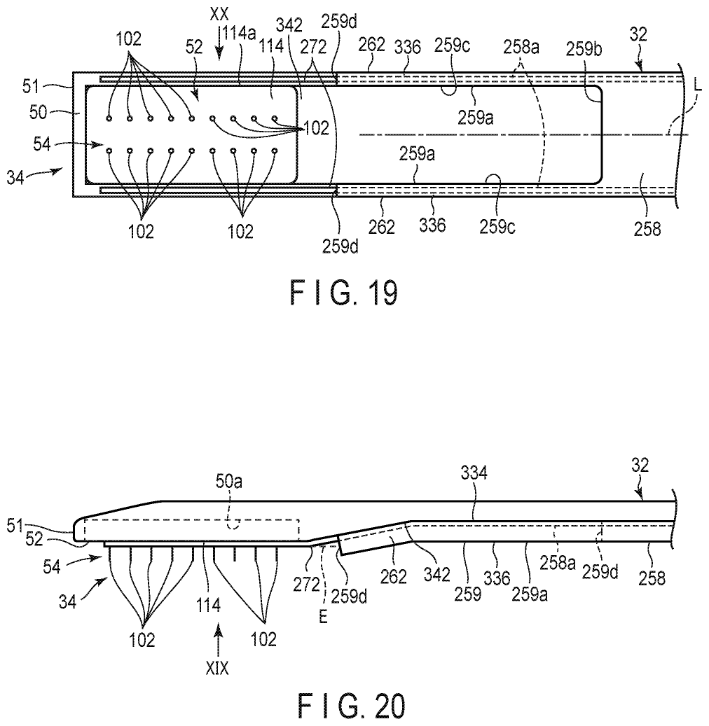

When the shape-memory portions 262 move from the position illustrated in FIG. 19 and FIG. 20 and advance beyond the distal ends of the guides 272 provided on the insertion section 32 and housing 50, the shape-memory portions 262 bend as illustrated in FIG. 17 and FIG. 18. At this time, the shape-memory portions 262 are opposed to the proximal edge 259b. Thus, when the cover portion 258 is in the first position, the cover portion 258 can surround the outer periphery of the needle section 54 in the same manner as described in the first embodiment. Accordingly, when the cover portion 258 is in the first position, the exposure of the tip ends 104 of the probes 102 can more exactly be prevented and the probes 102 can more exactly be protected by the structure of the present modification than in the example described in the third embodiment.

Note that in the example illustrated in FIG. 20 in which the cover portion 258 is in the second position, the extension line E of the guide 272 in the end effector 34 exists on the distal end 259d of the cover portion 258. At this time, it should suffice if the end effector 34 can pierce the tip ends 104 and output sections 56 of the needle section 54 into the biological tissue. In addition, in the end effector 34, the tip ends 104 and output sections 56 of the needle section 54 may be disposed in a position farther from the base 52 than the second end face (reference surface) 136 of the cover portion 258, for example, by adjusting the angle of the inclined surface 342 between the proximal portion of the housing 50 of the end effector 34 and the distal portion of the insertion section 32, or by adjusting the movable distance of the cover portion 258.

### Fourth Embodiment

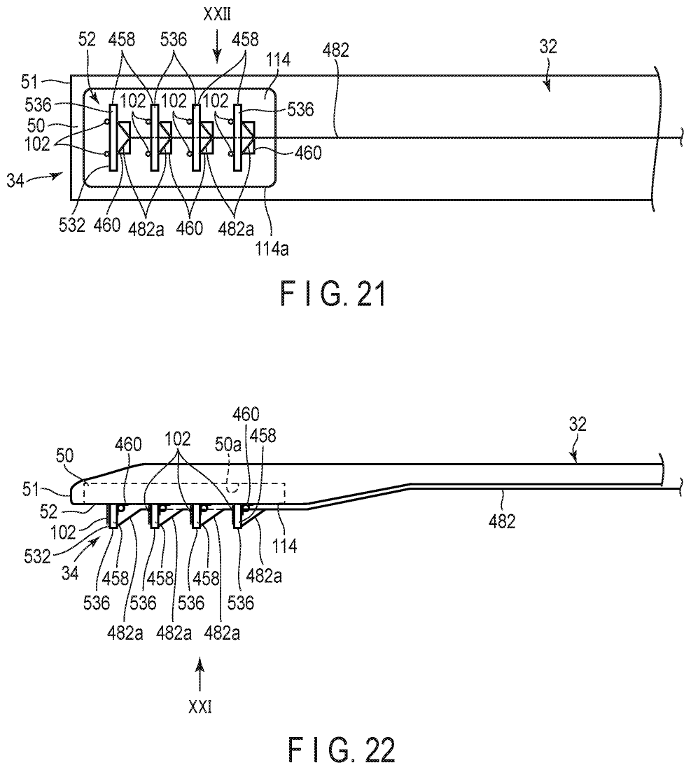

Next, a fourth embodiment will be described with reference to FIG. 21 to FIG. 24. This embodiment is a modification of the first embodiment including each modification, the second embodiment including each modification and the third embodiment including each modification. The same members or the members having the same functions as the members described in the first to third embodiments are denoted by like reference signs, and a detailed description thereof is omitted.

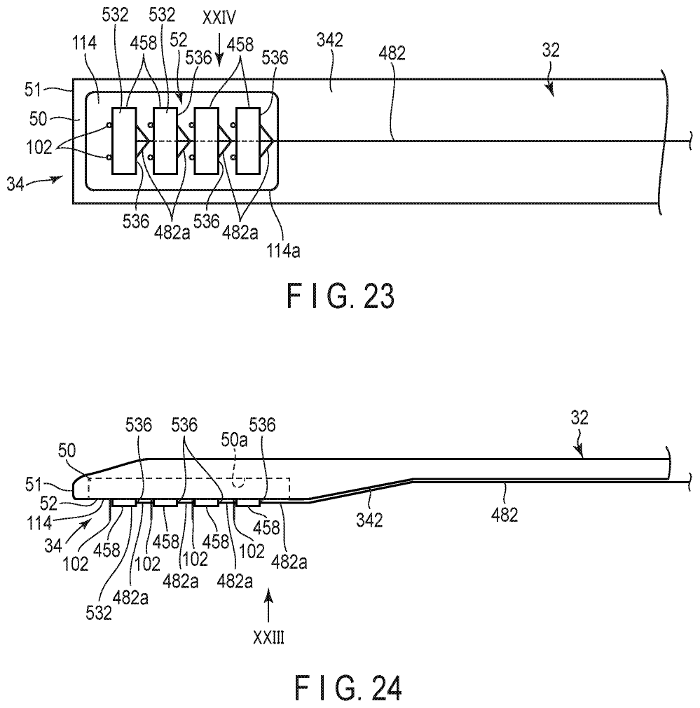

An energy treatment instrument 12 according to the present embodiment includes, like the first embodiment and second embodiment, an insertion section 32 in which a longitudinal axis L is defined, and an end effector 34. The end effector 34 includes a base 52, a needle section 54, energy output sections 56, and a plurality of cover portions 458. The cover portions 458 are provided on the base 52 and are movable relative to the base 52. The cover portions 458 are movable between a standing position illustrated in FIG. 21 and FIG. 22 and a lying position illustrated in FIG. 23 and FIG. 24. Thus, the cover portions 458 of the present embodiment are different from the cover portion 58 described in the first embodiment and the cover portion 258 described in the third embodiment with respect to the movable direction and the shape.

It is preferable that the cover portions 458 are formed of an electrically insulating resin material. Each cover portion 458 is formed in a plate shape and, in this embodiment, is coupled to the defining surface 114 of the base 52 by a hinge 460. For the purpose of simple description, a description is given of an example in which each Cover portion 458 is formed as a rectangular plate-shaped member. Each hinge 460 is rotated, for example, in a range between 0 degree and 90 degrees, or in a range less than 90 degrees.

For example, each cover portion 458 is disposed adjacent to a pair of probes 102 which are juxtaposed. Here, each cover portion 458 neighbors the proximal side of the probes 102 along the longitudinal axis L. A moving body 482, which extends to the proximal side along the longitudinal axis L of the insertion section 32, is coupled to each cover portion 458. A wire, for instance, is used as the moving body 482. A proximal end of the moving body 482 is coupled to the operation element 156 provided in the handle 36.

Each cover portion 458 is coupled via a support wire 482a to the moving body 482 which extends along the longitudinal axis L of the insertion section 32.

In addition, when the moving body 482 is positioned at the distal end along the longitudinal axis L of the slot 154 of the handle 36, the cover portions 458 are disposed in a first position illustrated in FIG. 21 and FIG. 22. In the first position illustrated in FIG. 21 and FIG. 22, the plate-shaped cover portions 458 stand, for example, in parallel to the probes 102. A reference surface (end portion) 536 (to be described later) of the cover portion 458 is disposed in a position which is equal to the tip end 104 of the needle section 54, or in a position (projection direction side) which projects from the tip end 104. Thus, when the cover portion 458 is in the first position, the reference surface 536 of the cover portion 458 is disposed in a position which is equal to the tip end 104 of the probe 102 of the needle section 54, or in a position which is farther from the defining surface 114 of the base 52 than the tip end 104. In this manner, when the cover portion 458 is in the first position, the end face (end portion) 536 of the cover portion 458 is disposed in the position which is equal to the tip end 104 of the needle section 54 or in the position which projects from the tip end 104, thereby protecting the tip ends 104 and output sections 56 of the needle section 54. Specifically, when the cover portions 458 are in the first position, the reference surface 536 of the cover portion 458 is disposed on the outside of the needle section 458 in such a state that the tip ends (needle points) 104 of the probes 102 are not exposed. At this time, each cover portion 458 collectively protects part of the probes 102.

When the moving body 482 is positioned at the proximal end along the longitudinal axis L of the slot 154 of the handle 36, each cover portion 458 is disposed in a second position illustrated in FIG. 23 and FIG. 24. The reference surface 536 of the cover portion 458 is disposed in a position (base 52 side) which is closer to the base 52 than the tip end 104 and energy output section 56 of the needle section 54. In the second position illustrated in FIG. 23 and FIG. 24, the plate-shaped cover portion 458 is fallen and laid relative to the probes 102. In this manner, when the cover portion 458 is in the second position, the end face (end portion) 536 of the cover portion 458 is evacuated from the tip end 104 and output section 56 of the needle section 54. In other words, when the cover portion 58 is in the second position, the tip ends (needle points) 104 and energy output sections 56 of the probes 102 project from the reference surface (end portion) 536 with respect to the cover portion 458. At this time, the tip ends (needle points) 104 and energy output sections 56 of the probes 102 are exposed from the cover portion 458. Thus, when the cover portions 458 are in the second position, the end effector 34 is capable of piercing the tip ends 104 and energy output sections 56 of the needle section 54 into the biological tissue. Although it is preferable that the cover portions 458 are moved at the same time, the cover portions 458 may be moved with time differences.

In addition, the cover portion 458 can advance and retreat (can move) between the first position and the second position.

The cover portion 458 includes the reference surface (reference edge) 536. The reference surface 536 is formed as an end portion, like the second end face 136 described in the first embodiment and the second end face 336 described in the second embodiment. The reference surface 536 may be a planar surface or a curved surface. Here, the height of the reference surface 536 of the cover portion 458 relative to the defining surface 114 of the base 52 is equal to or greater than the distance of the tip end 104 of the probe 102 from the defining surface 114 of the base 52. Thus, when the cover portions 458 are disposed in the position illustrated in FIG. 21 and FIG. 22, the passage from the entrance to the treatment target toward the treatment target is protected from the tip end 104 of each probe 102, and each probe 102 for use in a treatment is protected.

It is preferable that in the cover portion 458, a torsion spring (not shown) is provided as an urging body 74 for the hinge 460. In this case, the cover portion 458 is urged to the first position illustrated in FIG. 21 and FIG. 22.

Next, the function of the treatment system 10 according to the present embodiment will be described. A description of the parts described in the first embodiment, second embodiment and/or third embodiment is omitted unless otherwise necessary.

When the treatment target is accessed, the cover portions 458 are disposed in the first position illustrated in FIG. 21 and FIG. 22, relative to the housing 50 of the end effector 34 of the energy treatment instrument 12. At this time, the second end face (reference surface) 536 of the cover portion 458 is disposed in a position which is farther from the defining surface 114 of the base 52 than the tip ends 104 of the probes 102 of the needle section 54. Thus, when the end effector 34 of the energy treatment instrument 12 is inserted from the cavitas nasi CN toward the inferior turbinate IT of the treatment target, the tip end 104 of each probe 102 is prevented from abutting on the wall surface in the range from the cavitas nasi CN of the patient to the inferior turbinate IT of the treatment target. In addition, since the tip end 104 of each probe 102 is prevented from abutting on the wall surface in the range from the cavitas nasi CN of the patient to the inferior turbinate IT of the treatment target, a load is prevented from acting on each probe 102 before the treatment target is treated.

By moving the operation element 156 of the handle 36 shown in FIG. 1, the surgeon moves the cover portions 458 from the first position illustrated in FIG. 21 and FIG. 22 to the second position illustrated in FIG. 23 and FIG. 24, relative to the insertion section 32. At this time, as illustrated in FIG. 23 and FIG. 24, the second end face 536 of the cover portion 458 is separated from the tip end 104 of each probe 102 of the needle section 54, and the plate-shaped cover portion 458 is fallen down. Thus, when the cover portion 458 is in the second position (see FIG. 23 and FIG. 24), the reference surface 536 of the cover portion 458 is made closer to the defining surface 114 of the base 52 than when the cover portion 458 is in the first position. (see FIG. 21 and FIG. 22). Note that when the hinge 460 is provided with the torsion spring, the cover portion 458 is moved from the first position to the second position against the urging force of the torsion spring.

In addition, in the present embodiment, the projection length up to the tip end 104 of the probe 102 relative to the plate-shaped cover portion 458 is defined. Further, the projection length up to the energy output section 56 relative to the cover portion 458 is defined.

The surgeon puts a surface 532, which is included in the cover portion 458 of the end effector 34 that is in the second position and which is located on the opposite side to the surface facing the defining surface 114 of the base 52, into contact with the surface of the mucosal epithelial layer MEL of the inferior turbinate IT. Thus, in the state in which the surface 532 of the cover portion 458 is put in contact with the mucosal epithelial layer MEL, the tip ends 104 of the probes 102 reach the lamina propria mucosae LPM via the mucosal epithelial layer MEL. In particular, the energy output sections 56 are disposed in the upper layer including the shallow layer of the lamina propria mucosae LPM.

In addition, similarly as described in the first embodiment including each modification and the second embodiment including each modification, if the surgeon pushes the switch 15, an electric current is caused to flow in the shallow layer of the lamina propria mucosae LPM from the energy source 14 via the energy output sections 56, and the shallow layer of the lamina propria mucosae LPM of the inferior turbinate IT is properly cauterized.

When the surgeon largely moves the end effector 34, as in the case of pulling out the end effector 34 from the cavitas nasi CN, the surgeon moves the operation element 156 from the proximal end toward distal end of the slot 154. At this time, the moving body 482 advances along the longitudinal axis L of the insertion section 32. Thus, the cover portions 458 coupled to the moving body 482 are restored from the second position to the first position. In the state in which the cover portions 458 are in the first position, the surgeon moves the end effector 34 to a proper position.

According to the present embodiment, there can be provided the energy treatment instrument 12 which can protect the inside of the cavity at a time of accessing the treatment target. Furthermore, at a time of accessing the treatment target by the end effector 34, the probes 102 of the needle section 54 can be protected by the cover portions 458.

Besides, in the energy treatment instrument 12 according to the present embodiment, similarly as described in the first to third embodiments, there is no need to consider a load on the electrical path due to the movement of the needle section relative to the insertion section. Therefore, according to the present embodiment, there can be provided the energy treatment instrument 12 which can reduce the load acting on the electrical path.

When the hinge 460 is provided with the torsion spring, the cover portion 458 is moved from the second position to the first position by the urging force of the torsion spring in accordance with the movement of the moving body 482. In this manner, if the torsion spring is disposed on the hinge 460, when the position of the cover portion 458 is restored from the second position illustrated in FIG. 23 and FIG. 24 to the first position illustrated in FIG. 21 and FIG. 22, the position of the cover portion 458 can easily be restored by the urging force of the spring.

When the urging body such as the torsion spring is used, the moving body 482 may not be used. Each cover position 458 is movable only within a predetermined range by the hinge 460, and a load is prevented from acting on the probes 102 by the cover portion 458.

In the end effector 34 of the treatment instrument 12 according to the present embodiment, the urging body such as the torsion spring may or may not be used. In this case, the moving body 482 is used.

The example in which the cover portions 458 are formed in flat plate shapes is illustrated in FIG. 21 to FIG. 24 of this embodiment. However, each probe 102 may be individually protected by a cover portion having, for example, a halfpipe shape.

Although not illustrated, as described in the first embodiment, the insertion section 32 may be formed to be properly bendable.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment instrument comprising:
an insertion section in which a longitudinal axis is defined;
a base provided on a distal side along the longitudinal axis of the insertion section;
a needle section including a tip end projecting in a first direction relative to the base;
an output section provided toward a side of the base with respect to the tip end of the needle section, and configured to output energy to an outside of the needle section when supplied with energy; and
a cover portion including an end portion which is movable relative to the base, the cover portion being configured to move between:
a first position where the end portion of the cover portion is disposed in a position which is equal to the tip end of the needle section or in a position which projects from the tip end in the first direction, and the cover portion is configured to protect the tip end of the needle section and the output section, and
a second position where the end portion of the cover portion is evacuated from the tip end of the needle section and the output section.

2. The energy treatment instrument of claim 1, wherein when the cover portion is in the second position, a projection length of the output section relative to the end portion of the cover portion is defined.

3. The energy treatment instrument of claim 1, wherein
the needle section projects in a direction crossing the longitudinal axis of the insertion section, and
the cover portion is configured tO move in the direction crossing the longitudinal axis of the insertion section.

4. The energy treatment instrument of claim 3, wherein
the cover portion is provided outside the base,
the end portion of the cover portion is configured to move in a direction in which the needle section projects, and
when the cover portion is in the second position, the end portion of the cover portion is disposed in a position which is closer to the base than the tip end of the needle section and the output section, and the tip end and the output section project from the end portion of the cover portion.

5. The energy treatment instrument of claim 1, wherein
the needle section includes a plurality of probes supported on the base, and
the cover portion is configured to move in parallel to, or substantially in parallel to, an extending direction of the probes.

6. The energy treatment instrument of claim 5, comprising an urging body configured to move the cover portion in parallel to, or substantially in parallel to, the extending direction of the probes.

7. The energy treatment instrument of claim 1, wherein
the cover portion includes a distal portion which is configured to move along the longitudinal axis of the insertion section, and
when the cover portion is in the first position, the distal portion of the cover portion neighbors the needle section.

8. The energy treatment instrument of claim 7, wherein when the cover portion is in the second position, the distal portion of the cover portion is positioned more toward a proximal side along the longitudinal axis of the insertion section than when the cover portion is in the first position.

9. The energy treatment instrument of claim 1, wherein
the cover portion is coupled to the base by a hinge, and
when the cover portion is in the second position, the end portion of the cover portion is positioned closer to the base than when the cover portion is in the first position.

10. The energy treatment instrument of claim 1, comprising a housing including a guide configured to guide a movement of the cover portion relative to the base.

11. The energy treatment instrument of claim 1, comprising:
a housing in which the base is disposed; and
an urging body provided between the cover portion and the housing and configured to move the cover portion relative to the base.

12. The energy treatment instrument of claim 1, wherein the end portion of the cover portion is located at a distal end of a wall surface extending in a direction in which the needle section projects from the base.

13. The energy treatment instrument of claim 1, wherein
the base includes:
a defining surface which defines a length up to the tip end of the needle section, and
a support portion which supports a basal portion of the needle section, and
the end portion of the cover portion is located in a position which is closer to the tip end of the needle section than the defining surface.

14. The energy treatment instrument of claim 1, wherein
the needle section includes a plurality of probes, and
the cover portion is provided outside the needle section.

15. The energy treatment instrument of claim 1, wherein
the needle section includes a plurality of probes, and
the cover portion includes a plate-shaped member configured to individually protect the probes in a state in which the cover portion is in the first position.

16. The energy treatment instrument of claim 1, wherein
the needle section includes a plurality of probes, and
the cover portion is configured to collectively protect part of the probes.

17. The energy treatment instrument of claim 1, wherein
the output section is provided between the tip end of the needle section and the base, and
when the cover portion is in the second position, a position of the output section relative to the base or the end portion of the cover portion corresponds to a distance for reaching a lamina propria mucosae through a mucosal epithelial layer.

18. The energy treatment instrument of claim 1, comprising:
a handle provided on a proximal side of the insertion section; and
a rotary section provided between the insertion section and the handle and configured to rotate around the longitudinal axis of the insertion section,
wherein the insertion section, the base, the needle section, the output section, and the cover portion are rotatable as one piece around the longitudinal axis relative to the handle.

19. The energy treatment instrument of claim 1, comprising:
a handle provided on a proximal side of the insertion section; and
a moving body provided between the handle and the cover portion and configured to move along the longitudinal axis,
wherein the cover portion is configured to move relative to the base in accordance with a position to which the moving body is moved relative to the handle.

20. The energy treatment instrument of claim 19, comprising a lock mechanism provided on at least one of the handle and the moving body and configured to hold a position of the cover portion relative to the base.
